(19) United States
(12) Patent Application Publication
(10) Pub. No.: US 2024/0115196 A1
(43) Pub. Date: Apr. 11, 2024

(54) SEVERITY MAPS OF DENTAL CLINICAL FINDINGS

(71) Applicant: Align Technology, Inc., San Jose, CA (US)

(72) Inventors: Maayan Moshe, Ramat HaSharon (IL); Avi Kopelman, Palo Alto, CA (US); Moti Ben Dov, Tel Mond (IL); Ran Feder, Herzliya (IL); Yarden Matok, Jerusalem (IL); Ofer Saphier, Rechovot (IL)

(21) Appl. No.: 18/481,172

(22) Filed: Oct. 4, 2023

Related U.S. Application Data
(60) Provisional application No. 63/413,930, filed on Oct. 6, 2022.

(51) Int. Cl. A61B 5/00; A61B 5/103; A61C 13/34; G06T 7/00

(52) U.S. Cl. CPC A61B 5/4842; A61B 5/0088; A61B 5/1032; A61C 13/34; G06T 7/0016; G06T 2200/24; G06T 2207/10024; G06T 2207/10048; G06T 2207/20021; G06T 2207/20081; G06T 2207/30036

(57) ABSTRACT

A method includes receiving intraoral scan data of an intraoral cavity of a patient; processing the intraoral scan data to determine, for each dental condition of a plurality of dental conditions, whether the dental condition is detected for the patient and a severity of the dental condition; and presenting indications of the plurality of dental conditions together in a graphical user interface (GUI), wherein the indications show, for each dental condition of the plurality of dental conditions, whether the dental condition was detected for the patient and the severity of the dental condition.

### RELATED APPLICATIONS

This application claims the benefit under 35 U.S.C. § 119(e) of U.S. Provisional Application No. 63/413,930, filed Oct. 6, 2022, the entire content of which is incorporated by reference herein.

### TECHNICAL FIELD

Embodiments of the present disclosure relate to the field of dental diagnostics and, in particular, to a system and method for improving the process of diagnosing dental conditions.

### BACKGROUND

For a typical dental practice, a patient visits the dentist twice a year for a cleaning and an examination. A dental office may or may not generate a set of x-ray images of the patient's teeth during the patient visit. The dental hygienist additionally cleans the patient's teeth and notes any possible problem areas, which they convey to the dentist. The dentist then reviews the patient history, reviews the new x-rays (if any such x-rays were generated), and spends a few minutes examining the patient's teeth in a patient examination process. During the patient examination process, the dentist may follow a checklist of different areas to review. The examination can start with examining the patient's teeth for cavities, then reviewing existing restorations, then checking the patient's gums, then checking the patients' head, neck and mouth for pathologies or tumors, then checking the jaw joint, then checking the bite relationship and/or other orthodontic problems, and then checking any x-rays of the patient. Based on this review, the dentist makes a determination as to whether there are any dental conditions that need to be dealt with immediately and whether there are any other dental conditions that are not urgent but that should be dealt with eventually and/or that should be monitored. The dentist then needs to explain the identified dental conditions to the patient, talk to the patient about potential treatments, and convince the patient to make a decision on treatment for the patient's health. It can be challenging for the dentist to identify all problem dental conditions and convey the information about the dental conditions and their treatments to the patient in the short amount of time that the dentist has allotted for that patient.

### SUMMARY

A few example implementations of the present disclosure are described.

In a 1^{st} implementation, a method comprises: receiving an image of dental site of a patient; estimating a presence of one or more dental conditions in the image; estimating a severity level of each of the one or more dental conditions at one or more locations in the image; generating a severity map of the one or more dental conditions for the image based on the estimated presence of the one or more dental conditions and the estimated severity level of each of the one or more dental conditions at the one or more locations in the image; and projecting the severity map of the one or more dental conditions for the image onto a model of the dental site to generate a projected severity map of the one or more dental conditions.

A 2^{nd} implementation may further extend the 1^{st} implementation. In the 2^{nd} implementation, the model is a 3D model and the projected severity map is a 3D severity map, the method further comprising: presenting the 3D model of the dental site together with the 3D severity map of the one or more dental conditions in a graphical user interface (GUI).

A 3^{rd} implementation may further extend the 1^{st} or 2^{nd} implementation. In the 3^{rd} implementation, estimating the presence of the one or more dental conditions in the image comprises processing the image using one or more trained machined learning models, wherein each of the one or more trained machine learning models outputs a probability of the dental site containing the one or more dental conditions.

A 4^{th} implementation may further extend any of the 1^{st} through 3^{rd} implementations. In the 4^{th} implementation, the model is a 3D model of the dental site, the operations further comprising: receiving a plurality of intraoral scans of the dental site; and generating the 3D model of the dental site using the plurality of intraoral scans.

A 5^{th} implementation may further extend any of the 1^{st} through 4^{th} implementations. In the 5^{th} implementation, the method further comprises: receiving an intraoral scan of the dental site that is associated with the image; wherein the presence of the one or more dental conditions is estimated by inputting the image and the intraoral scan into a trained machine learning model that outputs a probability of the dental site containing the one or more dental conditions.

A 6^{th} implementation may further extend any of the 1^{st} through 5^{th} implementations. In the 6^{th} implementation, the one or more dental conditions are selected from a group consisting of caries, gum recession, gum inflammation, tooth wear, malocclusion, tooth crowding, tooth spacing, plaque, tooth stains, and tooth cracks.

A 7^{th} implementation may further extend any of the 1^{st} through 6^{th} implementations. In the 7^{th} implementation, the image comprises one of a two-dimensional (2D) color image generated by an intraoral scanner, a 2D near infrared image generated by the intraoral scanner, or a 2D color image generated by an image sensor of a device other than an intraoral scanner.

An 8^{th} implementation may further extend any of the 1^{st} through 7^{th} implementations. In the 8^{th} implementation, estimating the severity level of each of the one or more dental conditions at one or more locations in the image comprises: taking a derivative of the estimation with respect to input pixel intensities of the image, wherein severity is a function of the derivative.

A 9^{th} implementation may further extend any of the 1^{st} through 8^{th} implementations. In the 9^{th} implementation, estimating the severity level of each of the one or more dental conditions at one or more locations in the image comprises: erasing a region of the image; generating a modified image by processing the image with the erased region by a machine learning model trained to generate images of healthy dental sites, wherein the machine learning model fills in data for the erased region of the image; estimating the presence of the one or more dental conditions in the modified image; and determining a change in the estimation of the presence of the one or more dental conditions between the modified image and the image.

A 10^{th} implementation may further extend any of the 1^{st} through 9^{th} implementations. In the 10^{th} implementation, the machine learning model is a generative adversarial network (GAN).

An 11^{th} implementation may further extend any of the 1^{st} through 10^{th} implementations. In the 11^{th} implementation, estimating the severity level of each of the one or more dental conditions at one or more locations in the image comprises: inputting the image into a trained machine learning model that generates a feature vector representing the image and reconstructs the image from the feature vector, the trained machine learning model having been trained on images lacking the one or more dental conditions; determining differences between the image and the reconstructed image; and generating the severity map based on the differences between the image and the reconstructed image.

A 12^{th} implementation may further extend any of the 1^{st} through 11^{th} implementations. In the 12^{th} implementation, for each location of the image a degree of difference between the image and the reconstructed image at the location provides the severity level of one or more dental conditions at the location.

A 13^{th} implementation may further extend any of the 1^{st} through 12^{th} implementations. In the 13^{th} implementation, the image comprises a near infrared image and the one or more dental conditions comprises caries.

A 14^{th} implementation may further extend any of the 1^{st} through 13^{th} implementations. In the 14^{th} implementation, the image comprises a color image and the one or more dental conditions comprises gum inflammation.

A 15^{th} implementation may further extend any of the 1^{st} through 14^{th} implementations. In the 15^{th} implementation, the method further comprises: separately estimating, for each dental condition of a plurality of dental conditions, the presence of the dental condition in the image and the severity level of the dental condition at the one or more locations in the image; generating a plurality of severity maps for the image, wherein each of the plurality of severity maps is associated with a different one of the plurality of dental conditions; and projecting each of the plurality of severity maps onto the model of the dental site to generate a plurality of projected severity maps, each associated with the different one of the plurality of dental conditions.

A 16^{th} implementation may further extend the 15^{th} implementation. In the 16^{th} implementation, the method further comprises: generating a combined projected severity map based on the plurality of projected severity maps, wherein for each location of the one or more locations a combined severity level is determined based on severity levels of each of the plurality of projected severity maps at the location.

A 17^{th} implementation may further extend the 16^{th} implementation. In the 17^{th} implementation, the method further comprises: presenting the combined projected severity map overlaid on the model in a graphical user interface GUI); receiving a selection of a location of the one or more locations; and presenting separate data for one or more of the plurality of projected severity maps at the selected location.

An 18^{th} implementation may further extend any of the 15^{st} through 17^{th} implementations. In the 18^{th} implementation, the method further comprises: presenting the projected model of the dental site; receiving a selection of a dental condition of interest; and presenting a projected severity map of the plurality of projected severity maps that is associated with the selected dental condition of interest.

A 19^{th} implementation may further extend any of the 1^{st} through 18^{th} implementations. In the 19^{th} implementation, estimating the severity level of each of the one or more dental conditions at one or more locations in the image is performed using a gradient-weighted class activation mapping (Grad-CAM) algorithm.

A 20^{th} implementation may further extend any of the 1^{st} through 19^{th} implementations. In the 20^{th} implementation, the severity map comprises a heat map.

A 21^{st} implementation may further extend any of the 1^{st} through 20^{th} implementations. In the 21^{st} implementation, estimating the severity level of each of the one or more dental conditions at one or more locations in the image comprises: dividing the image into a plurality of overlapping patches, wherein each pixel of the image contributes to more than one of the plurality of overlapping patches; for each patch of the plurality of overlapping patches, processing the patch using a model that outputs a probability of that patch containing the one or more dental conditions; and for each pixel of the image, determining a severity level of the one or more dental conditions at the pixel based on a combination of probabilities of patches that include the pixel containing the one or more dental conditions.

A 22^{nd} implementation may further extend any of the 1^{st} through 21^{st} implementations. In the 22^{nd} implementation, the image comprises a first time stamp, the method further comprising: receiving a second image that comprises a second time stamp that predates the first time stamp; comparing the projected severity map to a second projected severity map generated for the second image to determine differences therebetween; and determining rates of change in severity levels for the one or more dental conditions based on a result of the comparing.

A 23^{rd} implementation may further extend the 22^{nd} implementation. In the 23^{rd} implementation, the method further comprises: identifying one or more locations at which a rate of change of the severity level exceeds a rate of change threshold; and flagging the one or more locations.

A 24^{th} implementation may further extend the 22^{nd} or 23^{rd} implementation. In the 24^{th} implementation, the method further comprises: determining a recommended frequency for the patient to visit a dentist based on at least one of the severity level or the rate of change of the severity level for the one or more dental conditions.

A 25^{th} implementation may further extend any of the 1^{st} through 24^{th} implementations. In the 25^{th} implementation, the method further comprises: identifying one or more locations at which the severity level exceeds a severity threshold; and flagging the one or more locations.

A 26^{th} implementation may further extend any of the 1^{st} through 25^{th} implementations. In the 26^{th} implementation, the method further comprises: generating, based on the estimated severity level of the one or more dental conditions at the one or more locations in the image, a recommendation for a dentist to assess the dental site.

A 27^{th} implementation may further extend any of the 1^{st} through 26^{th} implementations. In the 27^{th} implementation, the method further comprises: generating a plurality of additional projected severity maps of the one or more dental conditions from a plurality of additional images; and resolving differences between the projected severity map and the plurality of additional projected severity maps using a voting algorithm.

A 28^{th} implementation may further extend any of the 1^{st} through 27^{th} implementations. In the 28^{th} implementation, the model comprises a panoramic image of the dental site.

A 29^{th} implementation may further extend any of the 1^{st} through 28^{th} implementations. In the 29^{th} implementation, a system comprises: a memory; and a processing device operatively connected to the memory, the processing device to perform the method of any of the 1^{st} through 28^{th} implementations.

A 30^{th} implementation may further extend any of the 1^{st} through 28^{th} implementations. In the 39^{th} implementation, computer readable medium comprises instructions that, when executed by a processing device, cause the processing device to perform the method of any of the 1^{st} through 28^{th} implementations.

### BRIEF DESCRIPTION OF THE DRAWINGS

The present disclosure is illustrated by way of example, and not by way of limitation, in the figures of the accompanying drawings.

FIG. 1 illustrates an system for assessing clinical dental conditions in patients, in accordance with embodiments of the present disclosure.

FIGS. 2A-B illustrate a flow diagram for a method of analyzing a patient's teeth and gums, in accordance with embodiments of the present disclosure.

FIG. 3 illustrates a flow diagram for a method of analyzing a patient's dentition, in accordance with embodiments of the present disclosure.

FIG. 4A illustrates a flow diagram for a method of generating a severity map of one or more dental conditions, in accordance with embodiments of the present disclosure.

FIG. 4B illustrates a flow diagram for another method of generating a severity map of one or more dental conditions, in accordance with embodiments of the present disclosure.

FIG. 4C illustrates a flow diagram for another method of generating a severity map of one or more dental conditions, in accordance with embodiments of the present disclosure.

FIG. 4D illustrates a flow diagram for another method of generating a severity map of one or more dental conditions, in accordance with embodiments of the present disclosure.

FIG. 4E illustrates a flow diagram for another method of generating a severity map of one or more dental conditions, in accordance with embodiments of the present disclosure.

FIG. 5 illustrates a flow diagram for a method of determining a rate of change of one or more dental conditions, in accordance with embodiments of the present disclosure.

FIG. 6 illustrates a flow diagram for a method of flagging one or more regions containing one or more dental conditions, in accordance with embodiments of the present disclosure.

FIG. 7 illustrates a 3D model of a dental site with axes associated with many images of the dental site, in accordance with embodiments of the present disclosure.

FIG. 8A illustrates a 3D model of a dental site, in accordance with embodiments of the present disclosure.

FIG. 8B represents a NIRI image of the dental site modeled in FIG. 8A, in accordance with embodiments of the present disclosure.

FIG. 8C represents a color image of the dental site modeled in FIG. 8A, in accordance with embodiments of the present disclosure.

FIG. 9A represents a NIRI image of a dental site that includes a caries, in accordance with embodiments of the present disclosure.

FIG. 9B represents the NIRI image of FIG. 9A in which severity information of the caries has been automatically determined, in accordance with embodiments of the present disclosure.

FIGS. 10A-B represents color images of a dental site in which severity information of gun inflammation has been automatically determined, in accordance with embodiments of the present disclosure.

FIG. 11A represents a NIRI image of a dental site that includes a caries, in accordance with embodiments of the present disclosure.

FIG. 11B represents the NIRI image of FIG. 11A in which the caries have been labeled, in accordance with embodiments of the present disclosure.

FIG. 11C represents the NIRI image of FIG. 11A in which severity information of the caries has been automatically determined, in accordance with embodiments of the present disclosure.

FIG. 11D represents the NIRI image of FIG. 11A with an overlay of a caries severity map, in accordance with embodiments of the present disclosure.

FIG. 12 illustrates a block diagram of an example computing device, in accordance with embodiments of the present disclosure.

### DETAILED DESCRIPTION

Described herein are embodiments related to identifying and/or visualizing of clinical dental conditions on dental sites (e.g., on dental arches and/or teeth of patients). In embodiments, a dentist or doctor (terms used interchangeably herein) and/or their technicians may gather various information about a patient. Such information may include intraoral 3D scans of the patient's dental arches, infrared images of the patient's teeth, color 2D images of the patient's teeth and/or gums, and so on. Additionally, or alternatively, a patient may provide images of the their teeth and/or gums that they take using personal cameras and/or computing devices. The intraoral scans and/or images may be generated by an intraoral scanner, and at least some of the other data (e.g., other images) may be generated by one or more devices other than intraoral scanners. Additionally, different data may be gathered at different times. Each of the different data points may be useful for determining whether the patient has one or more types of dental conditions. In embodiments, gathered information may be used to generate a 3D model, a panoramic image, or other model of a dental site (e.g., of the patient's teeth and/or gums), to determine whether the dental site contains one or more clinical dental conditions, and/or to generate a severity map (or multiple severity maps) of the one or more clinical dental conditions. The severity map or maps may then be overlaid onto the 3D model, panoramic image, or other model of the dental site. The doctor may then review the severity map to quickly determine whether the patient has any clinical dental conditions, the locations of any clinical dental conditions, and the severities of any clinical dental conditions.

Prior techniques for identifying dental conditions at dental sites have relied upon pixel-level classification and/or image segmentation. These techniques generally work well for identifying regions of dental conditions that have well-defined boundaries. However, such techniques can be ineffective for determining the regions of a dental site that have dental conditions with boundaries that are not well-defined (e.g., where there is a gradient in severity rather than a stark contrast between locations having a dental condition and adjacent locations not having the dental condition. For example, if a patient has gingivitis, then there may be regions with severe inflammation, regions with lesser inflammation, regions with minor inflammation, and regions with no inflammation. For such situations, it can be difficult for a machine learning model that performs segmentation to determine which regions to classify as gingivitis regions and which regions to classify as healthy regions.

In embodiments, severity maps are determined for dental conditions that may not have well defined boundaries. Severity maps may also be generated for dental conditions that have well defined borders. In an example, severity maps may be generated for dental conditions such as caries, gum swelling/inflammation, and so on, which may not have well defined boundaries. Severity maps are generated in embodiments by using a trained machine learning model to determine whether an image of a dental site contains a representation of a dental condition (e.g., to perform an image level classification with regards to a dental condition being present or not being present in the image). Once the presence of a dental condition is identified, one or more operations are performed to generate a severity map of the dental condition on the image. Such operations may include, for example, running a Grad-CAM algorithm on the machine learning model and image, taking a derivative of dental condition probability with respect to pixel intensity for pixels of the image, and so on. The severity map generated for the image may then be projected onto a model (e.g., a 3D model of the dental site), and the projected severity map may be shown on the 3D model of the dental site using texture mapping or the like. The severity map may be a heat map, where changes in color indicate changes in severity of the dental condition.

Embodiments reduce an amount of time that it takes for a doctor to inspect a patient's jaw, while also increasing an accuracy of inspections. Embodiments also enable a doctor to visualize the boundaries of one or more dental conditions that may otherwise be difficult to visualize. Embodiments discussed herein may increase the speed and efficiency of diagnosing dental conditions of patients. Embodiments enable a dentist to determine, at a single glance of a graphical user interface (GUI), one or more dental conditions that might be of concern for a patient and the severity of such dental conditions. It enables the dentist to easily and quickly prioritize dental conditions to be addressed. Additionally, in embodiments processing logic may compare different identified dental conditions to determine any correlations between the different identified dental conditions. As a result, the processing logic may identify some dental conditions as symptoms of other underlying root cause dental conditions. For example, processing logic may identify tooth crowding and caries formation that results from the tooth crowding.

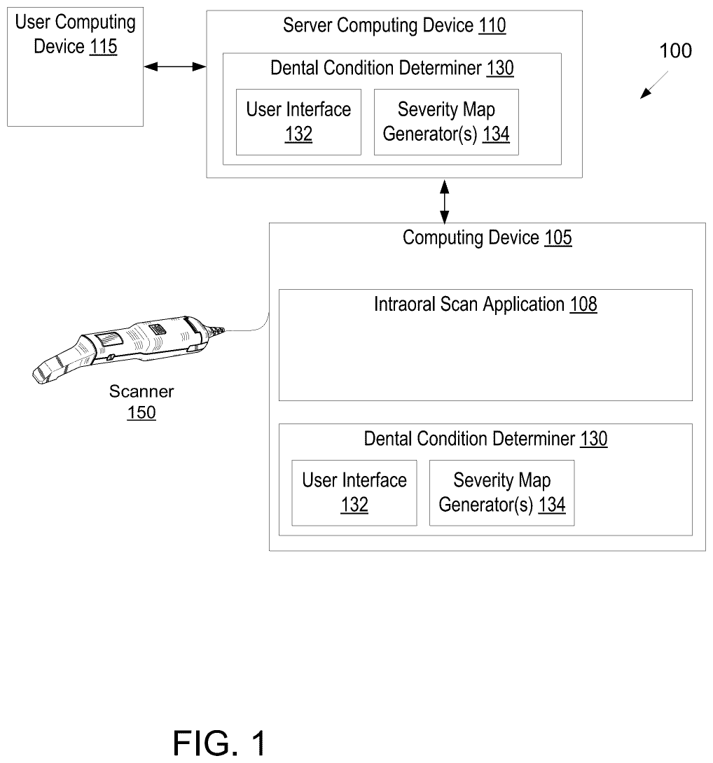

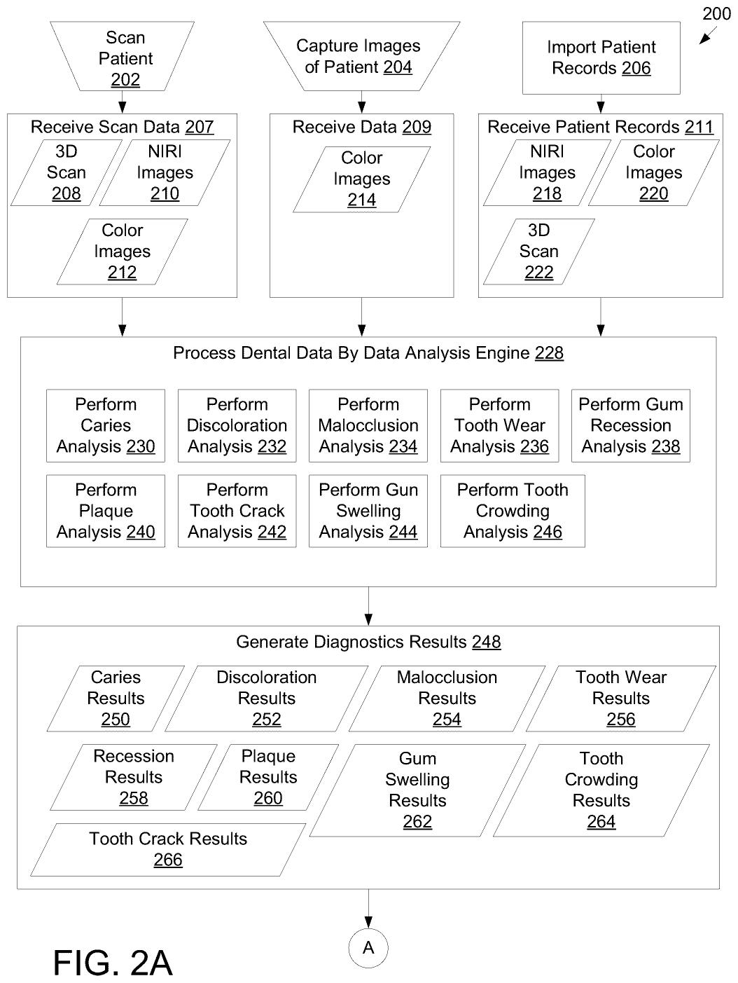

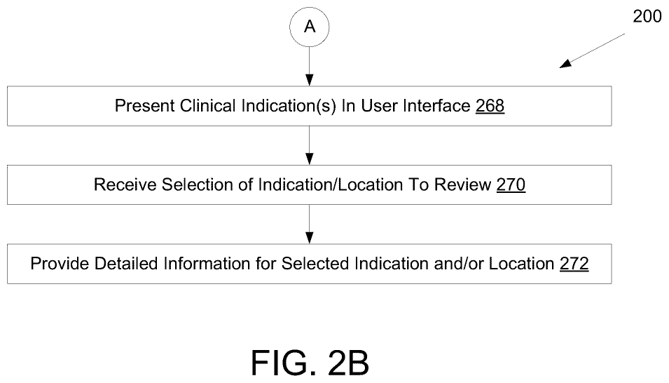

FIG. 1 illustrates one embodiment of a system 100 for performing intraoral scanning, generating a virtual three dimensional model of a dental site and/or performing dental diagnostics. In one embodiment, system 100 carries out one or more operations described below with reference to FIGS. 2A-6. System 100 includes a computing device 105 that may be coupled to a scanner 150. System 100 may additionally or alternatively include a server computing device 110, which may be connected to computing device 105 and/or a user computing device 115. Server computing device 110 may be connected to computing device 105 and/or user computing device 115 via a network. The network may be a local area network (LAN), a public wide area network (WAN) (e.g., the Internet), a private WAN (e.g., an intranet), or a combination thereof. The computing device 105 may be integrated into the scanner 150 in some embodiments to improve performance and mobility.

Computing devices 105, 110, 115 may each include a processing device, memory, secondary storage, one or more input devices (e.g., such as a keyboard, mouse, tablet, and so on), one or more output devices (e.g., a display, a printer, etc.), and/or other hardware components. Computing device 105, 110, 115 may be connected to and/or include a data store. The data store may be an internal data store, or an external data store that is connected to computing device 105, 110, 115 directly or via a network. Examples of network data stores include a storage area network (SAN), a network attached storage (NAS), and a storage service provided by a cloud computing service provider. The data store may include a file system, a database, or other data storage arrangement.

In some embodiments, a scanner 150 (also referred to as an intraoral scanner) for obtaining three-dimensional (3D) data of a dental site in a patient's oral cavity is operatively connected to the computing device 105. Scanner 150 may include a probe (e.g., a hand held probe) for optically capturing three dimensional structures. One example of such a scanner 150 is the iTero® intraoral digital scanner manufactured by Align Technology, Inc. Other examples of intraoral scanners include the 1M™ True Definition Scanner and the Apollo DI intraoral scanner and CEREC AC intraoral scanner manufactured by Sirona®.

The scanner 150 may be used to perform an intraoral scan of a patient's oral cavity. An intraoral scan application 108 running on computing device 105 may communicate with the scanner 150 to effectuate the intraoral scanning.

A dental practitioner (e.g., a dentist or dental technician) may use intraoral scanner 150 to perform an intraoral scan of a patient's oral cavity. Intraoral scan application 108 running on computing device 105 operatively connected to the intraoral scanner 150 may communicate with the scanner 150 to effectuate intraoral scanning and receive intraoral scan data (e.g., which may include intraoral images and/or intraoral scans). A result of the intraoral scanning may be a sequence of intraoral scans that have been discretely generated (e.g., by pressing on a “generate scan” button of the scanner for each image) or automatically generated (e.g., by pressing a “start scanning” button and moving the intraoral scanner around the oral cavity while multiple intraoral scans are generated). A further result of the intraoral scanning may be 2D images of the dental site, which may include 2D color images, 2D near infrared (NIRI) images, and so on. Each such image may be associated with a set of coordinates and/or a reference point that identifies a camera position and/or orientation of the camera that generated the image relative to the imaged dental site in some embodiments. In some embodiments, such coordinates/reference points are computed for one or more generated image.

An operator may start performing intraoral scanning at a first position in the oral cavity, and move the intraoral scanner within the oral cavity to various additional positions until intraoral scans and/or images have been generated for an entirety of one or more dental arches or until a particular dental site is fully scanned. In some embodiments, recording of intraoral scans and/or images may start automatically as teeth are detected or insertion into the oral cavity is detected and may automatically be paused or stopped as removal of the intraoral scanner from the oral cavity is detected.

According to an example, a user (e.g., a dental practitioner) may subject a patient to intraoral scanning. In doing so, the user may apply the intraoral scanner 150 to one or more patient intraoral locations. The scanning may be divided into one or more segments. As an example the segments may include a lower buccal region of the patient, a lower lingual region of the patient, a upper buccal region of the patient, an upper lingual region of the patient, one or more preparation teeth of the patient (e.g., teeth of the patient to which a dental device such as a crown or an orthodontic alignment device will be applied), one or more teeth which are contacts of preparation teeth (e.g., teeth not themselves subject to a dental device but which are located next to one or more such teeth or which interface with one or more such teeth upon mouth closure), and/or patient bite (e.g., scanning performed with closure of the patient's mouth with scan being directed towards an interface area of the patient's upper and lower teeth). In one embodiment, the segments include an upper dental arch segment, a lower dental arch segment and a patient bite segment. Via such scanner application, the scanner may generate intraoral scan data. The computing device 105 executing the intraoral scan application 108 may receive and store the intraoral scan data. The intraoral scan data may include two-dimensional (2D) intraoral images (e.g., color 2D images), three-dimensional intraoral scans (e.g., intraoral images with depth information such as monochrome height maps), intraoral images generated using infrared or near-infrared (NIRI) light, and/or intraoral images generated using ultraviolet light. The 2D color images, 3D scans, NIRI and/or infrared images and/or ultraviolet images may be generated by an intraoral scanner 150 capable of generating each of these types of intraoral scan data. Such intraoral scan data may be provided from the scanner 150 to the computing device 105 in the form of one or more points (e.g., one or more pixels and/or groups of pixels). For instance, the scanner 150 may provide such intraoral scan data as one or more point clouds.

A result of the intraoral scanning may be a sequence of intraoral images and/or scans that have been generated. Each intraoral scan may include x, y and z position information for one or more points on a surface of a scanned object. In one embodiment, each intraoral scan includes a height map of a surface of a scanned object. An operator may start a scanning operation with the scanner 150 at a first position in the oral cavity, move the scanner 150 within the oral cavity to a second position while the scanning is being performed, and then stop recording of intraoral scans. In some embodiments, recording may start automatically as the scanner identifies teeth. The scanner 150 may transmit the intraoral scans to the computing device 105. Computing device 105 may store the current intraoral scan data 135 from a current scanning session in a data store. The data store may additionally include past intraoral scan data, additional current dental data generated during a current patient visit (e.g., x-ray images, CBCT scan data, panoramic x-ray images, ultrasound data, color photos, and so on), additional past dental data generated during one or more prior patient visits (e.g., x-ray images, CBCT scan data, panoramic x-ray images, ultrasound data, color photos, and so on), and/or reference data. Alternatively, scanner 150 may be connected to another system that stores data in a data store. In such an embodiment, scanner 150 may not be connected to computing device 105.

In embodiments, intraoral scanning may be performed on a patient's oral cavity during a visitation of a dentist's office. The intraoral scanning may be performed, for example, as part of a semi-annual or annual dental health checkup. The intraoral scanning may be a full scan of the upper and lower dental arches, and may be performed in order to gather information for performing dental diagnostics. The dental information generated from the intraoral scanning may include 3D scan data, 2D color images, NIRI and/or infrared images, and/or ultraviolet images.

When a scan session is complete (e.g., all images for a dental site have been captured), intraoral scan application 108 may generate a virtual 3D model of the scanned dental site (e.g., of the upper and/or lower dental arches of the patient) from the intraoral scan data. To generate the virtual model or models (e.g., a separate model of the upper and lower dental arches), intraoral scan application 108 may register and “stitch” together the intraoral scans and/or images generated from the intraoral scanning session. In one embodiment, performing registration includes capturing 3D data of various points of a surface in multiple scans (views from a camera), and registering the scans by computing transformations between the images. The intraoral scans may then be integrated into a common reference frame by applying appropriate transformations to points of each registered intraoral scan.

In one embodiment, registration is performed for each pair of adjacent or overlapping intraoral scans. Registration algorithms may be carried out to register two adjacent intraoral scans for example, which essentially involves determination of the transformations which align one intraoral scan with the other. Registration may involve identifying multiple points in each intraoral scan (e.g., point clouds) of a pair of intraoral scans, surface fitting to the points of each intraoral scans, and using local searches around points to match points of the two adjacent intraoral scans. For example, the intraoral scan application 108 may match points, edges, curvature features, spin-point features, etc. of one intraoral scan with the closest points, edges, curvature features, spin-point features, etc. interpolated on the surface of the other intraoral scan, and iteratively minimize the distance between matched points. Registration may be repeated for each adjacent and/or overlapping scans to obtain transformations (e.g., rotations around one to three axes and translations within one to three planes) to a common reference frame. Using the determined transformations, the intraoral scan application may integrate the multiple intraoral scans into a first 3D model of the lower dental arch and a second 3D model of the upper dental arch.

The intraoral scan data may further include one or more intraoral scans showing a relationship of the upper dental arch to the lower dental arch. These intraoral scans may be usable to determine a patient bite and/or to determine occlusal contact information for the patient. The patient bite may include determined relationships between teeth in the upper dental arch and teeth in the lower dental arch.

The intraoral scan application 108 or another application may further register data from one or more other imaging modalities to the 3D model generated from the intraoral scan data. For example, processing logic may register 2D color images, NIRI images, and so on to the 3D model. Each of the different imaging modalities may contribute different information about the patient's dentition. For example, NIRI images may identify caries and color images may be used to add accurate color data to the 3D model, which is usable to determine tooth staining and/or gum inflammation. The registered intraoral data from the multiple imaging modalities may be presented together in the 3D model and/or side-by-side with one or more imaging modalities shown that reflect a zoomed in and/or highlighted section and/or orientation of the 3D model. The data from different imaging modalities may be provided as different layers, where each layer may be for a particular imaging modality. This may enable a doctor to turn on or off specific layers to visualize the dental arch with or without information from those particular imaging modalities.

In one embodiment, computing device 105 includes a dental condition determiner 130, which may include a user interface 132 and/or one or more severity map generators 134. The user interface 132 may be a graphical user interface and may include icons, buttons, graphics, menus, windows and so on for controlling and navigating the dental condition determiner 130.

In one embodiment, dental condition determiner 130 includes a separate severity map generator 134 for each type of dental condition that dental condition determiner 130 assesses. Each of the severity map generators 134 may be responsible for performing an analysis associated with a different type of dental condition. Alternatively, a single severity map generator 134 may generate severity maps for multiple dental conditions. Based on a result of the analysis, the severity map generator 134 may generate a severity map associated with a particular dental condition or dental conditions. In an example, severity map generators 134 may include severity map generators 134 for tooth cracks, gum recession, plaque, abrasion, attrition, abfractions, erosion, tooth stains, gum inflammation, and/or caries. Each of these severity map generators 134 may determine whether a particular type of dental condition or dental conditions is/are detected, may determine severity levels of the particular type(s) of dental condition(s) at various locations on a dental site, and may ultimately generate a severity map(s) of the dental condition(s) which can be overlaid onto a model (e.g., a 3D model) of the dental site. This may include projecting dental condition severity maps generated for multiple images onto the model using coordinates associated with a camera that generated the image at the time of image generation and/or by registering the images to the model as described above to determine transformation parameters, and then using the transformation parameters to project the severity maps onto the model. The severity maps for a same dental condition associated with multiple images may include pixels that maps to the same point on the model. The severity levels for that point from each of the severity maps may be combined in embodiments to determine a final severity level for the point, such as by averaging, using a voting algorithm, and so on.

In one embodiment, a severity map generator 134 is a combined severity map generator that generates a combined severity map that incorporates information from multiple discrete severity maps associated with different dental conditions. Multiple techniques for generating severity maps for dental conditions are discussed herein below, any one or more of which may be applied by the severity map generator(s) 134.

Each of the severity map generators 134 may perform one or more types of dental condition analyses using intraoral data (e.g., current intraoral scan data, past intraoral scan data, current NIRI images, past NIRI images, current color images, past color images and/or reference data). As a result, dental condition determiner 130 may determine multiple different dental conditions and severity levels of each of those types of identified dental conditions.

In one embodiment, the different types of dental conditions for which analyses are performed include tooth cracks, gum recession, tooth wear, occlusal contacts, gum inflammation, crowding and/or spacing of teeth and/or other malocclusions, plaque, tooth stains, and caries. Additional, fewer and/or alternative dental conditions may also be analyzed and reported. In embodiments, multiple different types of analyses are performed to determine presence and/or severity of one or more of the dental conditions. One type of analysis that may be performed is a point-in-time analysis that identifies the presence and/or severity levels of one or more dental conditions at a particular point-in-time based on data generated at that point-in-time. For example, a one or more NIRI images and/or color images of a dental arch that were generated during a same patient visit may be analyzed to determine whether, at a particular point-in-time, a patient's dental arch included any caries, gum recession, tooth wear, problem occlusion contacts, crowding, spacing or tooth gaps, plaque, tooth stains, gum inflammation, and/or tooth cracks.

Another type of analysis that may be performed is a time-based analysis that compares dental conditions at two or more points in time to determine changes in the dental conditions, progression of the dental conditions and/or rates of change of the dental conditions. For example, in embodiments a comparative analysis is performed to determine differences between severity maps of dental conditions generated from images taken at different points in time. The differences may be measured to determine an amount of change, and the amount of change together with the times at which the images that were used to generate the severity maps were taken may be used to determine a rate of change. This technique may be used, for example, to identify an amount of change and/or a rate of change for tooth wear, staining, plaque, crowding, spacing, gum recession, sum inflammation, caries development, tooth cracks, and so on.

In embodiments, dental condition determiner 130 additionally uses information of multiple different types of identified dental conditions and/or associated severity levels to determine correlations and/or cause and effect relationships between two or more of the identified dental conditions. Multiple dental conditions may be caused by the same underlying root cause. Additionally, some dental conditions may serve as an underlying root cause for other dental conditions. Treatment of the underlying root cause dental conditions may mitigate or halt further development of other dental conditions. For example, malocclusion (e.g., tooth crowding and/or tooth spacing or gaps), tooth wear and caries may all be identified for the same tooth or set of teeth. Dental condition determiner 130 may analyze these identified dental conditions that have a common, overlapping or adjacent area of interest, and determine a correlation or causal link between one or more of the dental conditions. In example, dental condition determiner 130 may determine that the caries and tooth wear for a particular group of teeth is caused by tooth crowding for that group of teeth. By performing orthodontic treatment for that group of teeth, the malocclusion may be corrected, which may prevent or reduce further caries progression and/or tooth wear for that group of teeth. In another example, plaque, tooth staining, and gum recession may be identified for a region of a dental arch. The tooth staining and gum recession may be symptoms of excessive plaque. The dental condition determiner 130 may determine that the plaque is an underlying cause for the tooth staining and/or gum recession.

In embodiments, currently identified dental conditions may be used by the dental condition determiner 130 to predict future dental conditions that are not presently indicated. Such analysis may be performed by inputting intraoral data (e.g., current intraoral data and/or past intraoral data) and/or the dental conditions identified from the intraoral data into a trained machine learning model that has been trained to predict future dental conditions based on current dental conditions and/or current dentition (e.g., current 3D surfaces of dental arches). The machine learning model may be any of the types of machine learning models discussed elsewhere herein. The machine learning model may output a probability map indicating predicted locations of dental conditions and/or types of dental conditions. Alternatively, the machine learning model may output a prediction of one or more future dental conditions without identifying where those dental conditions are predicted to be located.

In embodiments, one or more trained models are used by severity map generators 134 to perform at least some of the one or more dental condition analyses. The trained models may include physics-based models and/or machine learning models, for example. In one embodiment, a single model may be used to perform multiple different analyses (e.g., to identify any combination of tooth cracks, gum recession, tooth wear, occlusal contacts, crowding and/or spacing of teeth and/or other malocclusions, plaque, tooth stains, and/or caries). Additionally, or alternatively, different models may be used to identify different dental conditions. For example, a first model of a first severity map generator 134 may be used to identify tooth cracks, a second model of a second severity map generator 134 may be used to identify tooth wear, a third model of a third severity map generator 134 may be used to identify gum recession, a fourth model of a fourth severity map generator 134 may be used to identify problem occlusal contacts, a fifth model of a fifth severity map generator 134 may be used to identify crowding and/or spacing of teeth and/or other malocclusions, a sixth model of a sixth severity map generator 134 may be used to identify plaque, a seventh model of a seventh severity map generator 134 may be used to identify tooth stains, and/or an eighth model of an eighth severity map generator 134 may be used to identify caries. Alternatively, a single trained model may output dental condition estimates for multiple different dental conditions.

In one embodiment, intraoral data (e.g., images and/or intraoral scans) from one or more points in time are input into one or more trained machine learning models that have been trained to receive the intraoral data as an input and to output classifications of one or more types of dental conditions. The intraoral data that is input into the one or more trained machine learning model may include three-dimensional (3D) data and/or two-dimensional (2D) data. The intraoral data may include, for example, one or more 3D models of a dental arch, one or more projections of one or more 3D models of a dental arch onto one or more planes (optionally comprising height maps), near-infrared and/or infrared imaging data, color image(s), ultraviolet imaging data, intraoral scans, and so on. If data from multiple imaging modalities are used (e.g., 3D scan data, color images, and NIRI imaging data), then the data may be registered and/or stitched together so that the data is in a common reference frame and objects in the data are correctly positioned and oriented relative to objects in other data. One or more feature vectors may be input into the trained model, where the feature vectors include multiple channels of information for each point or pixel of an image. The multiple channels of information may include color channel information from a color image, depth channel information from intraoral scan data, a 3D model or a projected 3D model, intensity channel information from an x-ray image, and so on.

The trained machine learning model(s) may output a probability of the input data (e.g., image(s), intraoral scan(s), etc.) containing one or more types of clinical dental conditions. In one embodiment, the input data is input into multiple trained machine learning models, where each of the models outputs a probability that a different type of dental condition is identified. Alternatively, a single model may output probabilities of multiple different types of dental conditions being identified. Notably, in embodiments the trained machine learning model(s) outputs a single classification for an entire input, and does not output pixel or patch level classifications or perform segmentation.

Artificial neural networks (e.g., deep neural networks and convolutional neural networks) generally include a feature representation component with a classifier or regression layers that map features to a desired output space. A convolutional neural network (CNN), for example, hosts multiple layers of convolutional filters. Pooling is performed, and non-linearities may be addressed, at lower layers, on top of which a multi-layer perceptron is commonly appended, mapping top layer features extracted by the convolutional layers to decisions (e.g. classification outputs). Deep learning is a class of machine learning algorithms that use a cascade of multiple layers of nonlinear processing units for feature extraction and transformation. Each successive layer uses the output from the previous layer as input. Deep neural networks may learn in a supervised (e.g., classification) and/or unsupervised (e.g., pattern analysis) manner. Deep neural networks include a hierarchy of layers, where the different layers learn different levels of representations that correspond to different levels of abstraction. In deep learning, each level learns to transform its input data into a slightly more abstract and composite representation. In an image recognition application, for example, the raw input may be a matrix of pixels; the first representational layer may abstract the pixels and encode edges; the second layer may compose and encode arrangements of edges; the third layer may encode higher level shapes (e.g., teeth, lips, gums, etc.); and the fourth layer may recognize that the image contains a face or define a bounding box around teeth in the image. Notably, a deep learning process can learn which features to optimally place in which level on its own. The “deep” in “deep learning” refers to the number of layers through which the data is transformed. More precisely, deep learning systems have a substantial credit assignment path (CAP) depth. The CAP is the chain of transformations from input to output. CAPs describe potentially causal connections between input and output. For a feedforward neural network, the depth of the CAPs may be that of the network and may be the number of hidden layers plus one. For recurrent neural networks, in which a signal may propagate through a layer more than once, the CAP depth is potentially unlimited.

Training of a neural network may be achieved in a supervised learning manner, which involves feeding a training dataset consisting of labeled inputs through the network, observing its outputs, defining an error (by measuring the difference between the outputs and the label values), and using techniques such as deep gradient descent and backpropagation to tune the weights of the network across all its layers and nodes such that the error is minimized. In many applications, repeating this process across the many labeled inputs in the training dataset yields a network that can produce correct output when presented with inputs that are different than the ones present in the training dataset. In high-dimensional settings, such as large images, this generalization is achieved when a sufficiently large and diverse training dataset is made available.

To train the one or more machine learning models, a training dataset (or multiple training datasets, one for each of the machine learning models to be trained) containing hundreds, thousands, tens of thousands, hundreds of thousands or more images should be used to form a training dataset. In embodiments, up to millions of cases of patient dentition that include one or more labeled dental conditions such as cracked teeth, tooth wear, caries, gum recession, gum swelling, tooth stains, healthy teeth, healthy gums, and so on are used, where each case may include a final virtual 3D model of a dental arch (or other dental site such as a portion of a dental arch). The machine learning models may be trained to automatically classify intraoral scans and/or images, and the classification may be used to automatically determine presence and/or severity of dental conditions.

A training dataset may be gathered, where each data item in the training dataset may include an image (e.g., a color or NIRI image) of a dental site and an associated label indicating whether the dental site in the image contains a clinical dental condition and/or the type of class of the clinical dental condition at the dental site.

Additional data may include an intraoral scan and/or depth information associated with the image. For example, for each image (which may be a color or NIRI image), there may also be a corresponding color intraoral scan.

The result of this training is a function that can predict dental conditions directly from input images. In particular, the machine learning model(s) may be trained to generate a probability of the input images contain one or more dental conditions.

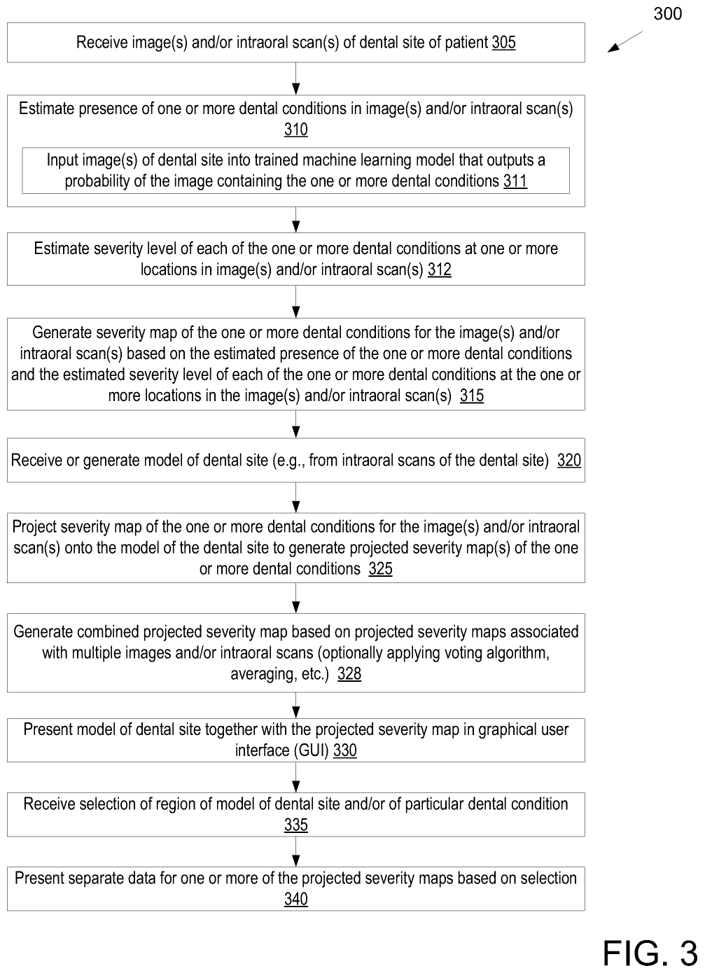

One or more operations may be performed (e.g., on a state of the machine learning model that generated the output and/or on the output of the machine learning model) by severity map generator(s) 134 to generate one or more severity maps of one or more dental conditions after such dental conditions have been identified in input data by the trained machine learning model or models. The various techniques for generating the severity maps are discussed below with reference to FIGS. 3-6.

The severity maps generated by severity map generator(s) 134 may be used to update one or more versions of the 3D model of the patient's upper and/or lower dental arches. In one embodiment, a different layer is generated for each dental condition class. A layer may be turned on to graphically illustrate areas of interest on the upper and/or lower dental arch that has been identified or flagged as having a particular dental condition.

If the severity maps were generated for one or more input 2D images (e.g., such as height maps in which pixel intensity represents height or depth), the severity maps may be projected onto points in the virtual 3D model. Accordingly, each point in the virtual 3D model may include probability information from severity maps of one or multiple different intraoral images that map to that point. In one embodiment, the probability information from the severity map is projected onto the 3D model as a texture. The updated 3D model may then include, for one or more points, vertexes or voxels of the 3D model (e.g., vertexes on a 3D mesh that represents the surface of the 3D model), multiple sets of severities, where different sets of severities associated with severity maps generated for different input images or other intraoral data may have different severity values.

Dental condition determiner 130 may modify the virtual 3D model by determining, for each point in the virtual 3D model, one or more severity values for that point. This may include using a voting function to determine one or more dental condition identifier and/or associated severity value for each point. Processing logic may determine the number of votes for each dental condition severity for a point, and may then classify the point as having a severity of a dental condition that receives the most votes for that dental condition. In some embodiments, points may be associated with multiple classes of dental conditions.

The one or more trained machine learning models that are used to identify, classify and/or determine a severity level for dental conditions may be neural networks such as deep neural networks or convolutional neural networks. Such machine learning models may be trained using supervised training in embodiments.

In some embodiments, computing device 105 provides generated 3D models of a patient's dental arches and/or intraoral scans and/or images of a patient's oral cavity to server computing device 110. Server computing device may then generate 3D models (if such models have not already been generated). Server computing device 110 may include a copy of dental condition determiner 130 (e.g., including user interface 132 and severity map generator(s) 134), which may generate the above described severity maps for one or more clinical dental conditions of a patient.

Server computing device 110 may additionally include images of a patients oral cavity (e.g., of the patient's teeth and/or gums) from user computing device 115. For example, user computing device may be or include a mobile phone, laptop computer, digital camera, tablet computer, desktop computer, and so on belonging to a patient. The patient may generate one or more color images of the patient's oral cavity using the camera of the user computing device 115, and may then transmit the image or images to server computing device 110. Server computing device 110 may have previously received one or more 3D models of the patient's dental arches. Alternatively, server computing device 110 may estimate a 3D model of the patient's dental arch or arches based on the image(s) received from user computing device.

Dental condition determiner 130 may determine whether the patient has one or more clinical dental conditions by analyzing the images and/or scans received from computing device 105 and/or user computing device 115. Severity map generator(s) 134 may generate one or more severity maps of dental conditions for the patient, and may present those severity maps via user interface 132. In embodiments, the severity maps may be overlaid on top of a model (e.g., a 3D model of a patient dental arch) for easy viewing by a patient and/or doctor. If dental conditions are identified, then dental condition determiner 130 may communicate with user computing device 115 to recommend or schedule a visit with their dentist and/or with computing device 105 to notify the dentist of a severity of the dental condition(s).

In embodiments, the user interface 132 may output a combined severity map overlaid onto a 3D model of a dental site (e.g., as a texture). The combined severity map may indicate, for each region or location of the dental site, an overall combined severity of some or all detected dental conditions at the location.

From the overlay of the combined severity map, a dentist may select any of the types of dental classes that they are interested in and/or may select a particular location on the dental site. For example, the dentist may select any one of tooth cracks, caries, gum recession, tooth wear, occlusion, crowding/spacing, plaque, gum swelling, and/or tooth stains. This may cause the combined severity map to be replaced with a severity map associated with the selected type of clinical dental condition. In another example, a doctor may select a particular location (e.g., that has a high severity level). Detailed information for that location may than be presented, including a breakdown of the severity levels of each of the types of dental conditions that were detected at the selected location.

In embodiments, the severity map or maps help a doctor to quickly detect dental conditions and their respective severity levels, help the doctor to make better judgments about treatment of dental conditions, and further help the doctor in communicating with a patient that patient's dental conditions and possible treatments. This makes the process of identifying, diagnosing, and treating dental conditions easier and more efficient. The doctor may select any of the dental conditions to determine prognosis of that condition as it exists in the present and how it will likely progress into the future.

In embodiments, a doctor may customize dental conditions and/or areas of interest by adding emphasis or notes to specific dental conditions and/or areas of interest. For example, a patient may complain of a particular tooth aching. The doctor may highlight that particular tooth on the 3D model of the dental arches. Dental conditions that are found that are associated with the particular highlighted or selected tooth may then be shown in a dental diagnostics summary. In a further example, a doctor may select a particular tooth (e.g., lower left molar), and a dental diagnostics summary may be updated by modifying the severity results to be specific for that selected tooth. For example, if for the selected tooth an issue was found for caries and a possible issue was found for tooth stains, then the dental diagnostics summary would be updated to show no issues found for tooth wear, occlusion, crowding/spacing, plaque, tooth cracks, and gum recession, to show a potential issue found for tooth stains and to show an issue found for caries. This may help a doctor to quickly identify possible root causes for the pain that the patient complained of for the specific tooth that was selected. The doctor may then select a different tooth to get a summary of dental issues for that other tooth. Additionally, the doctor may select a dental arch, a quadrant of a dental arch, or a set of teeth, and the dental diagnostics summary may be updated to show the dental conditions associated with the selected set of teeth, quadrant of a dental arch, and/or dental arch.

FIGS. 2A-6 illustrate flow diagrams of methods performed to assess severity levels of one or more dental conditions of a patient, in accordance with embodiments of the present disclosure. These methods may be performed by processing logic that comprises hardware (e.g., circuitry, dedicated logic, programmable logic, microcode, etc.), software (such as instructions run on a processing device), or a combination thereof. In one embodiment, processing logic corresponds to computing device 105 and/or of server computing device 110 of FIG. 1.

FIGS. 2A-B illustrate a flow diagram for a method 200 of analyzing a patient's teeth and gums, in accordance with embodiments of the present disclosure. At block 202, a doctor or dental practitioner scans a patient's intraoral cavity. At block 207, processing logic receives the scan data from the scan performed at block 202. The scan data may include 3D scan data (e.g., color or monochrome 3D scan data 208, which may be received in the form of point clouds, height maps, images, or other data types) and/or one or more 3D models of the patient's dental arches generated based on the scan data. The scan data may further include NIRI images 210 and/or color images 212. The 3D scan data 208, NIRI images 210 and/or color images 212 may all be generated by an intraoral scanner.

At block 204, a doctor, patient or dental practitioner may generate additional patient data. At bock 209, processing logic receives the additional patient data. The additional patient data may include images (e.g., 2D color images) generated by a device other than an intraoral scanner, such as an imaging device of the patient or an imaging device in a dentist office. At block 206, processing logic may import patient records for the patient being scanned. At block 211, processing logic may receive the imported patient records. The imported patient records may include historical patient data such as historical NIRI images 218, color images 220, 3D scan data or 3D models generated from such 3D scan data 222, and/or other information.

At block 228, processing logic processes the received dental data (including the data received at blocks 208, 209 and/or 211) using one or more data analysis engine (e.g., severity map generators 134 of FIG. 1) to identify the presence of one or more types of dental conditions and/or severity levels for the one or more detected dental conditions. Processing logic may perform a caries analysis 230, a discoloration analysis 232, a malocclusion analysis 234, a tooth wear analysis 236, a gum recession analysis 238, a plaque analysis 240, a tooth crack analysis 242, a gum swelling analysis 244 and/or a tooth crowding analysis 246 (and/or tooth spacing analysis), as discussed herein above. The various analyses may include point-in-time analyses as well as time-dependent analyses that are based on data from multiple different times. For example, older scan data may be compared to recent scan data to determine whether dental conditions have improved, stayed the same, worsened, etc. Additionally, trajectories for the various dental conditions at various areas of interest where the dental conditions were identified may be determined and projected into the future and/or past. In some embodiments, the outputs of one or more dental health analyzers are used (alone or together with intraoral data) as inputs to other dental health analyzers. For example, the presence or absence of malocclusion may be used as in input into a dental health analyzer that performs the caries analysis 230 and/or the dental health analyzer that performs plaque analysis 240.

At block 248, processing logic generates diagnostics results based on an outcome of the dental condition analyses performed at block 228. Processing logic may generate caries results 250, discoloration results 252, malocclusion results 254, tooth wear results 256, gum recession results 258, plaque results 260, gum swelling results 262, tooth crowding and/or spacing results 264 and/or tooth crack results 266. The diagnostics results may include severity maps for each of the types of dental conditions. Each of the severity map generators 134 may ultimately generate a severity map for a particular type of dental condition, where the severity map can be overlaid onto a model of the patient's dental arch.

In some embodiments, severity maps for one or more dental conditions generated from data associated with different times may be compared to determine information such as to whether a dental condition at an location has improved, has stayed the same, or has worsened, indications as to the rapidity with which the dental condition has improved or worsened, an acceleration in the improvement or worsening of the dental condition, and so on. An expected rate of change may have been determined (e.g., automatically or with doctor input), and the measured rate of change for a dental condition at a location may be compared to the expected rate of change. Differences between the expected rate of change and the measured rate of change may be recorded and included in the diagnostics results. Each of the diagnostics results may be automatically assigned a code on dental procedures and nomenclature (CDT) code or other procedural code for health and adjunctive services provided in dentistry. Each of the diagnostics results may automatically be assigned an appropriate insurance code and related financial information.

At block 268, processing logic presents clinical indications of the dental condition analysis results in a user interface (e.g., of a dental condition determiner). The clinical indications may additionally be automatically added to a patient chart. For example, a patient chart may automatically be updated to identify each identified dental condition, a tooth and/or gum region affected by the dental condition, a severity level of the dental condition, and/or other information about an area of interest (AOI) at which the dental condition was identified. The doctor may add notes about the dental conditions as well, which may also be added to the patient chart.

The information presented in the user interface may include qualitative results and/or quantitative results of the various analyses. In embodiments, a dental diagnostics summary is shown that includes high level results, but that does not include low level details or detailed information underlying the high level results. All of the results of the analyses may be presented together in a unified view that improves clinical efficiency and provides for improved communication between the doctor and patient about the patient's oral health and how best to treat dental conditions.

The information presented in the user interface may include information identifying one or more new dental conditions that were detected in the current or most recent patient visit but not in the prior patient visit. The information presented in the user interface may include information identifying one or more preexisting dental conditions that have improved between the prior patient visit and the current or most recent patient visit. The information presented in the user interface may include information identifying one or more preexisting dental conditions that have worsened between the prior patient visit and the current or most recent patient visit. The information presented in the user interface may include information identifying one or more preexisting dental conditions that have not changed between the prior patient visit and the current or most recent patient visit.

At block 270, processing logic receives a selection of an indication to review and/or of a location to review. For example, a doctor may select caries indications to review, or may select a location on a dental arch with a particularly high severity level. At block 272, processing logic provides detailed information for the selected indication and/or location. For example, if the doctor selected to view caries, then a combined severity map may be replaced with a caries severity map. If the doctor selected a particular location on the patient's dental arch, then detailed information about the severity of multiple different dental conditions detected at that location may be displayed.

FIG. 3 illustrates a flow diagram for a method 300 of analyzing a patient's dentition, in accordance with embodiments of the present disclosure. At block 305 of method 300, processing logic receives one or more intraoral scans and/or images of a patient's oral cavity. The images may include, for example color images, NIRI images, and so on.

At block 310, processing logic processes the intraoral scan(s) and/or image(s) to determine, for each dental condition of a plurality of dental conditions, whether the dental condition is detected for the patient. In one embodiment, at block 311 processing logic inputs the image(s) and/or intraoral scan(s) into one or more trained machine learning models. The data input into the trained machine learning model(s) may include images (which may be cropped), intraoral scans, 3D surface data and/or projections of 3D models onto 2D planes in embodiments. The one or more trained machine learning models may be trained to receive the input data and to output classifications of detected dental conditions. In one embodiment, the output of the one or more trained machine learning models includes one or more probabilities of dental conditions being present in the input data. For example, a first probability of caries being present, a second probability of gum swelling being present, and so on may be output.

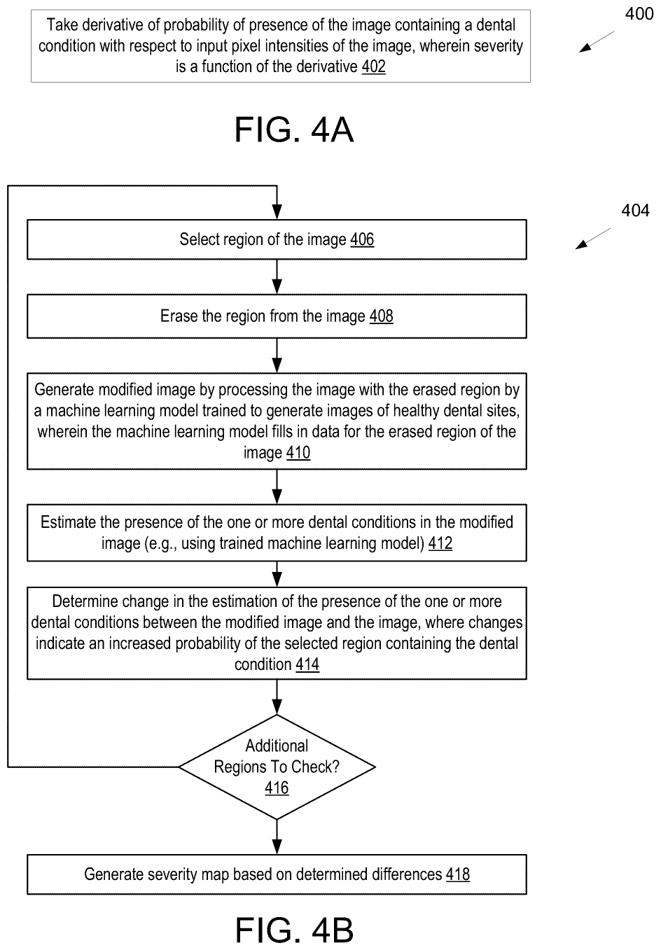

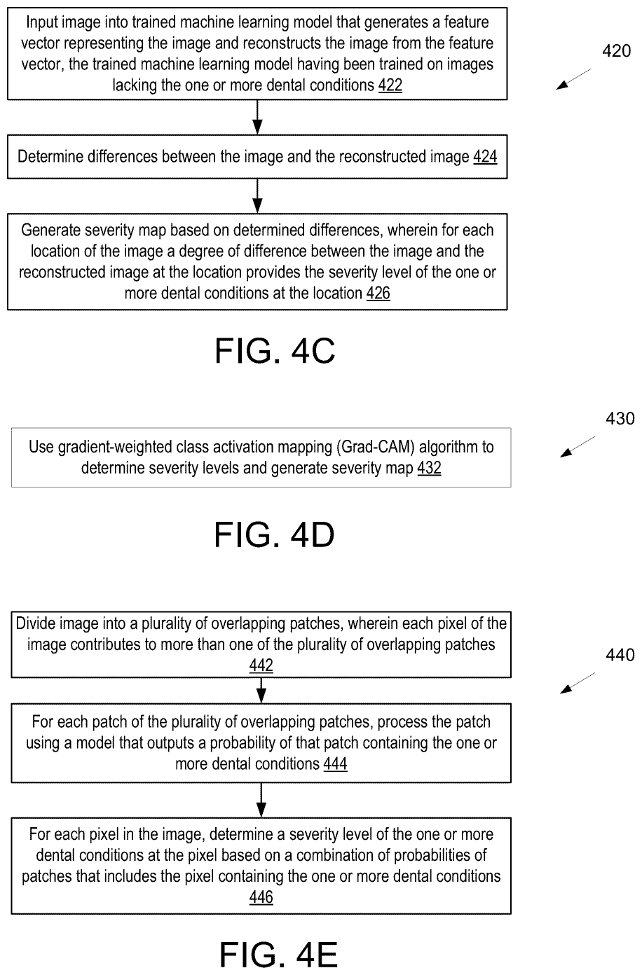

At block 312, processing logic estimates a severity level of each of the one or more dental conditions at one or more locations in the image(s) and/or intraoral scan(s). At block 315, processing logic generates a severity map of the one or more dental conditions for the image(s) and/or intraoral scan(s) based on the estimated presence of the one or more dental conditions and the estimated severity level of each of the one or more dental conditions at the one or more locations in the image(s) and/or intraoral scan(s). Determination of the severity levels of dental conditions at locations on the dental site and generation of the severity map may be performed using one or more of the techniques discussed below with reference to FIGS. 4A-6 in embodiments. In embodiments, the operations of block 312 and 315 are performed together (e.g., as a single operation).

At block 320, processing logic may generate a model or models (e.g., a 3D model and/or 2D panoramic model) of one or more dental arch of the patient using scan data. Alternatively, the model(s) may already have been generated.

At block 325, processing logic projects the severity map(s) of the one or more dental images that was generated for the image(s) and/or intraoral scan(s) onto the model(s) of the dental site to generate one or more projected severity maps of the one or more dental conditions.

In one embodiment, at block 328 processing logic generates a combined projected severity map based on projected severity maps associated with multiple images and/or intraoral scans. A separate combined projected severity map may be generated for each dental condition in one embodiment. In embodiments, processing logic may apply a voting algorithm to generate the combined severity map. For example, multiple projected severity maps (each generated for a different 2D image) may contain points that project to the same 3D point on a 3D model of the patient's dental arch. These different projected severity maps may contain different dental condition classifiers for that point and/or different severity levels of a dental condition for that point. A voting algorithm may be applied to enable each of the projected severity maps to essentially vote on a severity of dental condition at the point. In another embodiment, processing logic may apply an average (e.g., a weighted average) to combine the severity levels for the point associated with each of the projected severity maps.

In one embodiment, a combined severity map may take into consideration severities of multiple different dental conditions. An overall severity level may be determined for each point based on severities of multiple different dental conditions detected at the point. For example, severity levels of multiple dental conditions at a point may be aggregated to result in a combined or aggregate severity level for the point. Some dental conditions may be more concerning or problematic than others. Accordingly, in some embodiments the combined severity map is based on a weighted combination of severities of multiple different dental conditions. The weights applied to each dental condition severity level may be based on the relative importance associated with that dental condition relative to other dental conditions in some embodiments.

Any of the severity maps discussed herein above may be a heat map, where severity levels are associated with different colors and/or fill patterns. For example, red may be associated with a high severity level, green or blue may be associated with a low or zero severity level, and color shades in between may be associated with intermediate severity levels. This enables a doctor to assess a patient's dental health at a quick glance of the severity map or maps.

At block 330, processing logic presents one or more severity map (e.g., the combined projected severity map) together with the model of the dental site in a graphical user interface or other display.

At block 335, processing logic may receive a selection of a region of the model of the dental site and/or of a particular dental condition. At block 340, processing logic presents separate data for the one or more projected severity maps based on the selection.

FIG. 4A illustrates a flow diagram for a method 400 of generating a severity map of one or more dental conditions, in accordance with embodiments of the present disclosure. Method 400 may be performed at blocks 312 and/or 315 of method 300 in embodiments. Prior to execution of method 400, an image may have been processed by a trained machine learning model, which may have output a probability of the image containing a dental condition.

At block 402 of method 400, processing logic computes a derivative of the probability of the image containing the dental condition with respect to input pixel intensities of the image that was processed by the trained machine learning model. For example, the image may be modified multiple times by changing pixel intensities of one or more pixels of the image. Each modified image may be processed by the trained machine learning model, and a probability of each of the modified images including the dental condition may be computed. The greater the change in the probability of a modified image containing the dental condition, the higher the contribution of the pixel that was modified to that determined probability, and the greater the severity of the dental condition associated with that pixel. Accordingly, the severity of the dental condition at a pixel can be computed as a function of the derivative of the probability of the image containing the dental condition with respect to pixel intensity. By modifying many pixels of the image by various amounts and testing the modified images using the trained machine learning model, a severity map can be generated, where severities at each pixel location are based on a derivative of probability of a dental condition being found with respect to pixel intensity at the pixel location. A high derivative would indicate high severity, while a low derivative would indicate a low severity in embodiments.

FIG. 4B illustrates a flow diagram for another method 404 of generating a severity map of one or more dental conditions, in accordance with embodiments of the present disclosure. Method 404 may be performed at blocks 312 and/or 315 of method 300 in embodiments. Prior to execution of method 404, an image may have been processed by a trained machine learning model, which may have output a probability of the image containing a dental condition.

At block 406 of method 404, processing logic selects a region of the image. At block 408, processing logic may erase the selected region from the image. At block 410, processing logic may then generate a modified image by processing the image with the erased region by a machine learning model trained to generate images of healthy dental sites. The machine learning model may generate a new image in which the erased portion of the image has been filled in with synthetic image data. Since the model was trained using only healthy teeth and gums, the portion of the image generated by the machine learning model is of healthy teeth and gums (e.g., that does not have any clinical dental conditions).

In one embodiment, a generative adversarial network (GAN) (e.g., a generator of a GAN) is used for one or more machine learning models that generate the modified version of the image. A GAN is a class of artificial intelligence system that uses two artificial neural networks contesting with each other in a zero-sum game framework. The GAN includes a first artificial neural network (generator) that generates candidates and a second artificial neural network (discriminator) that evaluates the generated candidates. The GAN learns to map from a latent space to a particular data distribution of interest (a data distribution of changes to input images that are indistinguishable from photographs to the human eye), while the discriminative network discriminates between instances from a training dataset and candidates produced by the generator. The generative network's training objective is to increase the error rate of the discriminative network (e.g., to fool the discriminator network by producing novel synthesized instances that appear to have come from the training dataset). The generative network and the discriminator network are co-trained, and the generative network learns to generate images that are increasingly more difficult for the discriminative network to distinguish from real images (from the training dataset) while the discriminative network at the same time learns to be better able to distinguish between synthesized images and images from the training dataset. The two networks of the GAN are trained once they reach equilibrium. The GAN may include a generator network that generates artificial intraoral images and a discriminator network that segments the artificial intraoral images. In embodiments, the discriminator network may be a MobileNet.

In one embodiment, the machine learning model is a conditional generative adversarial (cGAN) network, such as pix2pix. These networks not only learn the mapping from input image to output image, but also learn a loss function to train this mapping. GANs are generative models that learn a mapping from random noise vector z to output image y, G:z→y. In contrast, conditional GANs learn a mapping from observed image x and random noise vector z, to y, G:{x, z}→y. The generator G is trained to produce outputs that cannot be distinguished from “real” images by an adversarially trained discriminator, D, which is trained to do as well as possible at detecting the generator's “fakes”. The generator may include a U-net or encoder-decoder architecture in embodiments. The discriminator may include a MobileNet architecture in embodiments. An example of a cGAN machine learning architecture that may be used is the pix2pix architecture described in Isola, Phillip, et al. “Image-to-image translation with conditional adversarial networks.” arXiv preprint (2017).

At block 412, processing logic estimates the presence of the one or more dental conditions in the modified image. This may include inputting the modified image into the trained machine learning model trained to determine whether images contain one or more dental conditions. At block 414, processing logic determines whether there has been any change in the estimation of the one or more dental conditions between the modified image and the original image (and/or one or more other modified images). For example, a probability of an image containing a dental condition may be output by the trained machine learning model for the original image and for the modified image. A difference in the probability may then be determined between the outputs for the two images. If the probability of the image containing the dental condition has decreased for the modified image as compared to the original (or previous) image, then a determination can be made that the region that was erased contributed to the higher probability of the dental condition for the original (or previous image). The degree of difference may be indicative of the severity level of the dental condition for that region. For example, a function may be used to receives an amount of change to the probability as an input and that outputs a severity level of the dental condition based on the amount of change to the probability.

At block 416, processing logic determines whether there are additional regions to check. If so, the method returns to block 406 and a new region of the image (or the modified image) is selected for modification. The remaining operations may then be performed to determine a severity level associated with the additional region. This process may be repeated until all regions of the image have been tested or until another stopping condition is reached. If at block 416 no further regions are to be checked, then the method proceeds to block 418.

At block 418, processing logic generates a severity map based on the determined differences and/or severity levels determined for each of the tested regions.

FIG. 4C illustrates a flow diagram for another method 420 of generating a severity map of one or more dental conditions, in accordance with embodiments of the present disclosure. Method 420 may be performed at blocks 312 and/or 315 of method 300 in embodiments. Prior to execution of method 420, an image may have been processed by a trained machine learning model, which may have output a probability of the image containing a dental condition.

At block 422 of method 420, processing logic inputs the image into a trained machine learning model that generates a feature vector representing the image and reconstructs the image from the feature vector. The feature vector may be a reduced feature vector that includes less information than the original image. The machine learning model may have been trained on images of healthy teeth and gums (e.g., that lacked the one or more dental conditions). Accordingly, the reconstructed image that is generated from the reduced feature vector will be an image of a dental site that lacks the dental conditions.

In one embodiment, the machine learning model is an encoder-decoder network (also referred to as an autoencoder). An encoder-decoder model includes an encoder that maps input data to a different, lower dimensional (e.g., compressed) feature representation and a decoder that takes the feature representation and recreates (to the best of its ability) the input data from the feature representation.

In one embodiment, a U-net architecture is used for the machine learning model. A U-net is a type of deep neural network that combines an encoder and decoder together, with appropriate concatenations between them, to capture both local and global features. The encoder is a series of convolutional layers that increase the number of channels while reducing the height and width when processing from inputs to outputs, while the decoder increases the height and width and reduces the number of channels. Layers from the encoder with the same image height and width may be concatenated with outputs from the decoder. Any or all of the convolutional layers from encoder and decoder may use traditional or depth-wise separable convolutions.

At block 424, processing logic compares the reconstructed image with the original image. Any differences between the reconstructed image and the original image can be attributable to dental conditions since the dental conditions do not get generated from the compressed feature representation (since the machine learning model was not trained on images with dental conditions), or in the reconstructed image. Accordingly, regions (e.g., pixels) at which differences between the images are determined can be assigned severity levels based on the degree of difference at or surrounding those regions.

At block 426, processing logic generates a severity map based on the determined differences between the original image and the reconstructed image. For each location of the image a degree of difference between the image and the reconstructed image at the location can provide the severity level of the one or more dental conditions at the location.

FIG. 4D illustrates a flow diagram for another method 430 of generating a severity map of one or more dental conditions, in accordance with embodiments of the present disclosure. Method 430 may be performed at blocks 312 and/or 315 of method 300 in embodiments. Prior to execution of method 430, an image may have been processed by a trained machine learning model, which may have output a probability of the image containing a dental condition.

At block 432 of method 430, processing logic uses a gradient-weighted class activation mapping (Grad-CAM) algorithm (or similar algorithm that generates a class activation mapping, such as high res CAM) to generate the severity map. The basic idea behind Grad-CAM is to exploit the spatial information that is preserved through convolutional layers, in order to understand which parts of an input image were important for a classification decision. Such algorithms make Convolutional Neural Network (CNN)-based models more transparent by visualizing the regions of input that contribute to for predictions from these models. Gradient-weighted Class Activation Mapping (Grad-CAM), uses the class-specific gradient information flowing into the final convolutional layer of a CNN to produce a coarse localization map of the important regions in the image. Grad-CAM is a strict generalization of Class Activation Mapping. Unlike CAM, Grad-CAM requires no re-training and is broadly applicable to any CNN-based architectures. Grad-CAM is a form of post-hoc attention, meaning that it is a method for producing heat maps that is applied to an already-trained neural network after training is complete and the parameters are fixed. Previously, Grad-CAM algorithms have been used solely for understanding how machine learning models are trained, and have not been used to determine clinical information. By contrast, embodiments apply the Grad-CAM algorithm to determine clinical information about images of dental sites. In particular, embodiments apply the Grad-CAM algorithm to generate a severity map of one or more dental conditions for images of dental sites.

FIG. 4E illustrates a flow diagram for another method 440 of generating a severity map of one or more dental conditions, in accordance with embodiments of the present disclosure. Method 440 may be performed at blocks 312 and/or 315 of method 300 in embodiments. Prior to execution of method 440, an image may have been processed by a trained machine learning model, which may have output a probability of the image containing a dental condition.

At block 442 of method 440, processing logic divides an image into a plurality of overlapping patches. Each pixel of the image may contribute to more than one of the overlapping patches. At block 444, processing logic processes each of the patches using a trained machine learning model that was trained to determine whether input images contain one or more dental conditions.

At block 446, for each pixel in the image, processing logic determines a severity level of the one or more dental conditions at the pixel based on a combination of the probability of patches that include the pixel containing the one or more dental conditions. The output of block 444 may be a set of values for each pixel, where each pixel is associated with a tuple representing the output probability of a dental condition for each patch that included the pixel. For example, a pixel may have been included in 10 patches, and 1 of those patches may have been classified as 90% chance of containing a dental condition while the other 9 patches were classified as 0% chance of containing the dental condition. The probabilities may be combined at block 436 (e.g., via an averaging process) to determine a severity level of the dental condition for the pixel. In the above example, the pixel would be assigned a very low severity value.

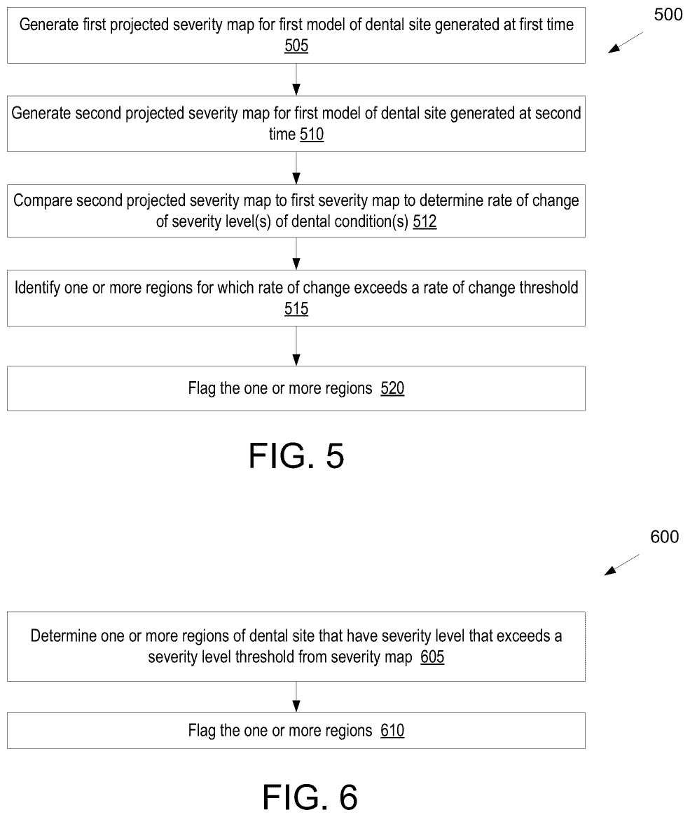

FIG. 5 illustrates a flow diagram for a method 500 of determining a rate of change of one or more dental conditions, in accordance with embodiments of the present disclosure. At block 505 of method 500, processing logic generates a first projected severity map for a first model of a dental site. The first model of the dental site may have been generated based on first data collected at a first time, and the first projected severity map may have been generated from images generated at the first time. At block 510, processing logic generates a second projected severity map for the first model or a second model of the dental site generated based on data collected at a second time. Processing logic may generate the second severity map from images generated at the second time. Further severity maps may also be generated based on images generated at other times.

At block 512, processing logic compares the second projected severity map to the first severity map. Additionally, any additional severity maps generated based on data from other times may be compared to the first and/or second severity maps. Based on the comparison, processing logic may determine changes between the severity maps, and may determine rates of change of the severity levels of one or more dental conditions based on the changes and the times at which the respective images used to generate the severity maps were generated. Processing logic may generate a heat map of a rate of change of severity of dental conditions in some embodiments.

At block 515, processing logic may identify one or more regions of the dental site for which the rate of change exceeds a rate of change threshold. At block 520, processing logic may flag the one or more regions for inspection by a doctor. In one embodiment, processing logic determines a suggested frequency of patient visits and/or a next visit based on an existing severity map and/or a determined rate of change of severity for one or more dental conditions. For example, processing logic may suggest an increased frequency of patient visits to a dentist for instances where a severity of a dental condition is increasing and/or is high.

FIG. 6 illustrates a flow diagram for a method 600 of flagging one or more regions containing one or more dental conditions, in accordance with embodiments of the present disclosure. At block 605 of method 600, processing logic determines one or more regions of a dental site that have a severity level that exceeds a severity level threshold from a severity map. At block 610, processing logic flags the one or more regions for inspection by a doctor.

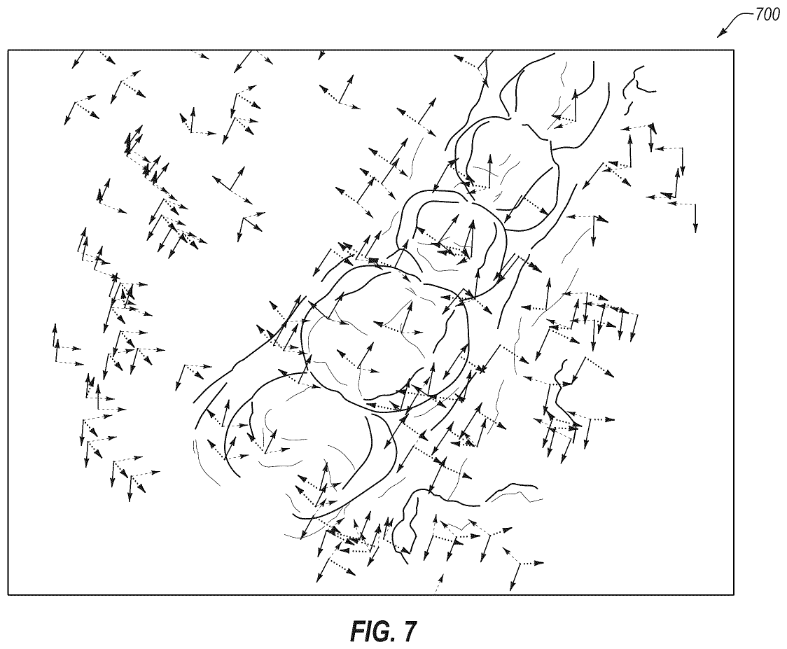

FIG. 7 illustrates a 3D model of a dental site 700 with axes associated with many images of the dental site, in accordance with embodiments of the present disclosure. For each image of a dental site, an origin of a camera that generated the image may be determined relative to the dental site. This origin may be, for example, assumed to be taken from a pinhole camera relative to the 3D model of the dental site 700. In FIG. 7 each pinhole camera position and orientation is represented by a coordinate frame. The position and orientation of the camera that generated an image may be used to project the image, and the severity map generated for the image, onto the 3D model. Similar techniques may be used to project images and associated severity maps onto panoramic views/images and/or flattened models of dental arches.

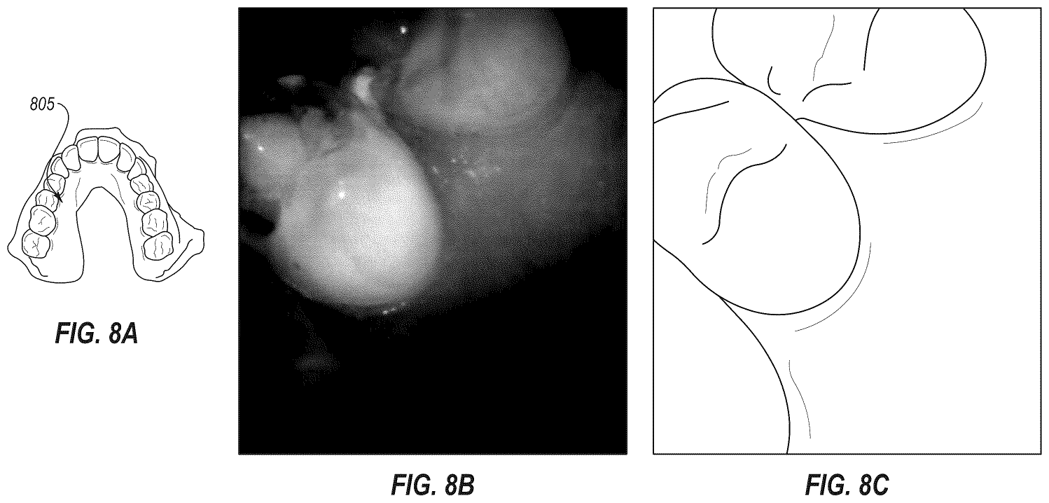

FIG. 8A illustrates a 3D model of a dental site, in accordance with embodiments of the present disclosure. A position 805 of a camera that generated an image relative to the 3D model of the dental site may be determined and shown.

FIG. 8B represents a NIRI image of the dental site modeled in FIG. 8A, in accordance with embodiments of the present disclosure. FIG. 8C represents a color image of the dental site modeled in FIG. 8A, in accordance with embodiments of the present disclosure. The NIRI image of FIG. 8B and the color image of FIG. 8C may have been generated from the position 805 of the camera. Images that show dental conditions may be labeled to generate a training data set for training a machine learning model to classify images as containing or not containing dental conditions in embodiments. In embodiments, a machine learning model is trained to make image-level classifications (as opposed to pixel-level classifications, patch-level classifications, or image segmentation). Labeling of images with image-level classifications to generate a training data set is much less work-intensive than labeling of images with pixel-level classifications. Accordingly, embodiments enable machine learning models to be trained to identify dental conditions and ultimately for generation of severity maps with far less expenditure of resources than traditional techniques.

In embodiments, a machine learning model is trained to perform image-level classification of dental conditions. The machine learning model may then be used to determine if there is a clinical finding in an image. Once the machine learning model predicts a clinical finding for an image, processing logic may take a gradient loss with respect to each image pixel intensity, or perform a similar process such as Grad-CAM, as described above.

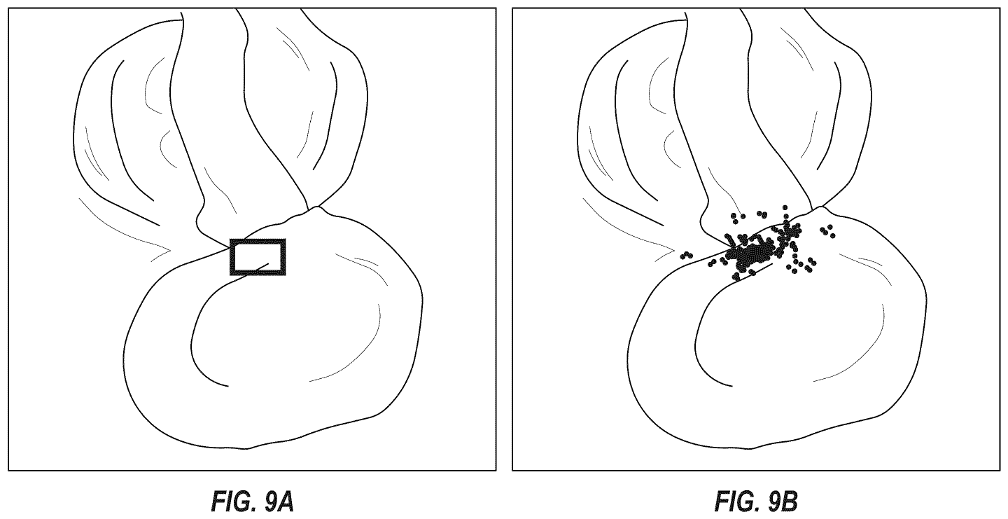

FIG. 9A represents a NIRI image of a dental site that includes a caries, in accordance with embodiments of the present disclosure. A bounding box indicates where a caries is located.

FIG. 9B represents the NIRI image of FIG. 9A in which severity information of the caries has been automatically determined by determining a gradient of the image intensities with respect to the loss, in accordance with embodiments of the present disclosure. As shown a gradient map indicates the presence of the caries or lesion.

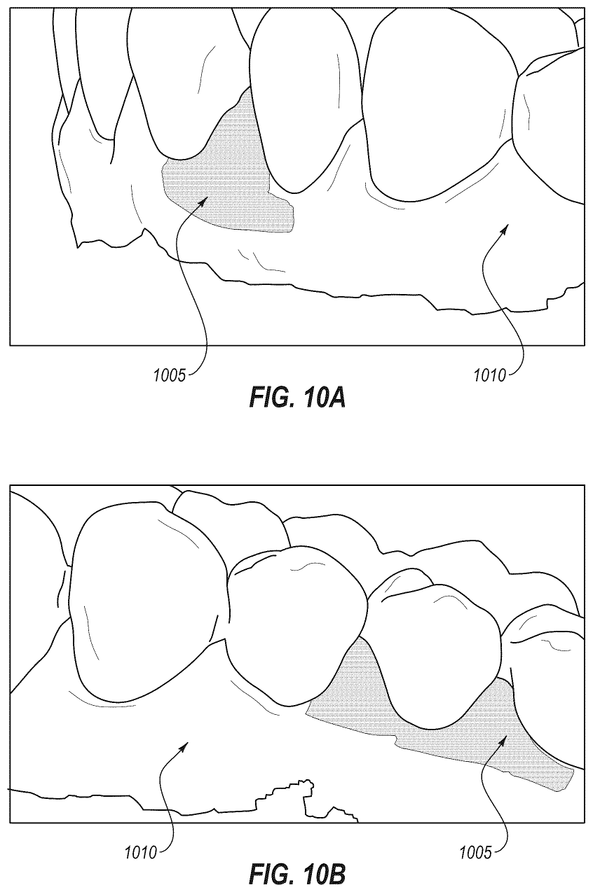

FIGS. 10A-B represent color images of a dental site in which severity information of gum inflammation has been automatically determined, in accordance with embodiments of the present disclosure. In each image, gum inflammation is shown with a bright color (e.g., a bright red or pink color), while healthy gums are shown with a less bright (e.g., darker) pink color.

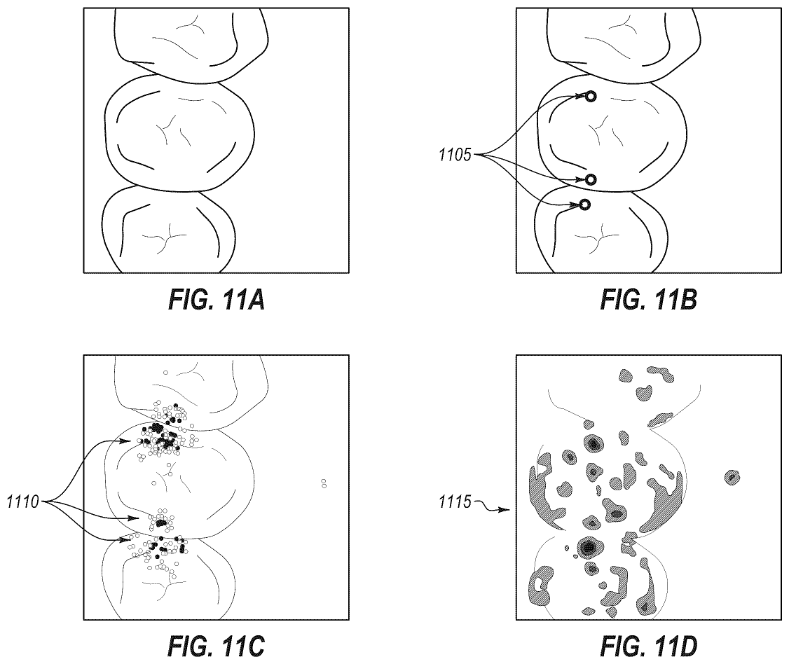

FIG. 11A represents a NIRI image of a dental site that includes a caries, in accordance with embodiments of the present disclosure.

FIG. 11B represents the NIRI image of FIG. 11A in which the caries 1105 have been labeled (caries are represented with circles), in accordance with embodiments of the present disclosure.

FIG. 11C represents the NIRI image of FIG. 11A in which severity information 1110 of the caries has been automatically determined as a gradient (e.g., a first type of severity map), in accordance with embodiments of the present disclosure.

FIG. 11D represents the NIRI image of FIG. 11A with an overlay of a caries severity map 1115 (e.g., as generated by using a Grad-CAM algorithm), in accordance with embodiments of the present disclosure.

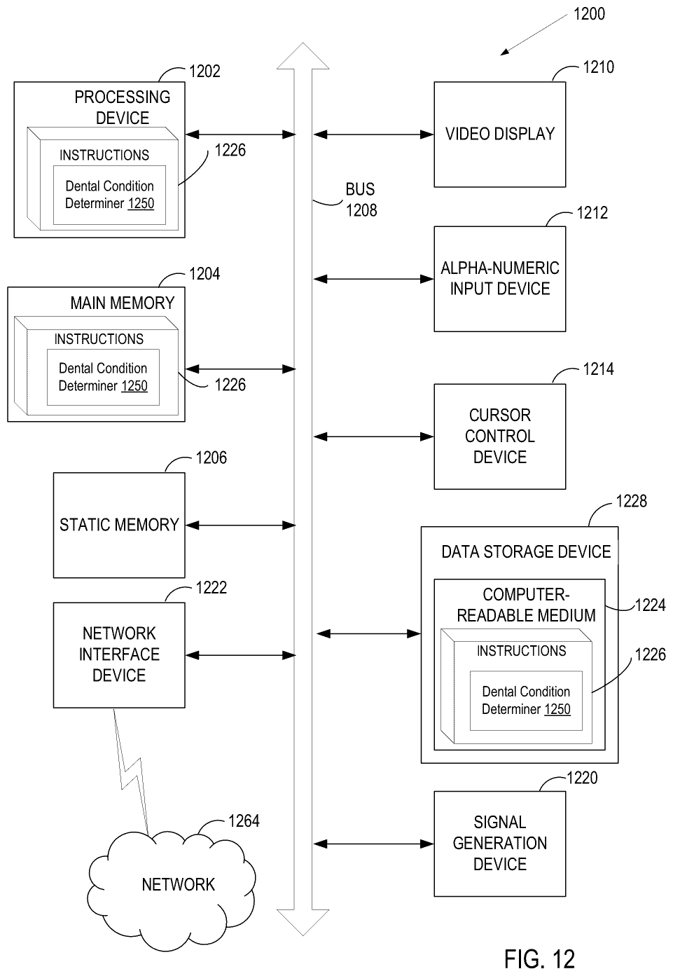

FIG. 12 illustrates a diagrammatic representation of a machine in the example form of a computing device 1200 within which a set of instructions, for causing the machine to perform any one or more of the methodologies discussed herein, may be executed. In alternative embodiments, the machine may be connected (e.g., networked) to other machines in a Local Area Network (LAN), an intranet, an extranet, or the Internet. The machine may operate in the capacity of a server or a client machine in a client-server network environment, or as a peer machine in a peer-to-peer (or distributed) network environment. The machine may be a personal computer (PC), a tablet computer, a set-top box (STB), a Personal Digital Assistant (PDA), a cellular telephone, a web appliance, a server, a network router, switch or bridge, or any machine capable of executing a set of instructions (sequential or otherwise) that specify actions to be taken by that machine. Further, while only a single machine is illustrated, the term “machine” shall also be taken to include any collection of machines (e.g., computers) that individually or jointly execute a set (or multiple sets) of instructions to perform any one or more of the methodologies discussed herein. In one embodiment, the computing device 1200 corresponds to computing device 105 and/or server computing device 110 of FIG. 1.

The example computing device 1200 includes a processing device 1202, a main memory 1204 (e.g., read-only memory (ROM), flash memory, dynamic random access memory (DRAM) such as synchronous DRAM (SDRAM), etc.), a static memory 1206 (e.g., flash memory, static random access memory (SRAM), etc.), and a secondary memory (e.g., a data storage device 1228), which communicate with each other via a bus 1208.

Processing device 1202 represents one or more general-purpose processors such as a microprocessor, central processing unit, or the like. More particularly, the processing device 1202 may be a complex instruction set computing (CISC) microprocessor, reduced instruction set computing (RISC) microprocessor, very long instruction word (VLIW) microprocessor, processor implementing other instruction sets, or processors implementing a combination of instruction sets. Processing device 1202 may also be one or more special-purpose processing devices such as an application specific integrated circuit (ASIC), a field programmable gate array (FPGA), a digital signal processor (DSP), network processor, or the like. Processing device 1202 is configured to execute the processing logic (instructions 1226) for performing operations and steps discussed herein.

The computing device 1200 may further include a network interface device 1222 for communicating with a network 1264. The computing device 1200 also may include a video display unit 1210 (e.g., a liquid crystal display (LCD) or a cathode ray tube (CRT)), an alphanumeric input device 1212 (e.g., a keyboard), a cursor control device 1214 (e.g., a mouse), and a signal generation device 1220 (e.g., a speaker).

The data storage device 1228 may include a machine-readable storage medium (or more specifically a non-transitory computer-readable storage medium) 1224 on which is stored one or more sets of instructions 1226 embodying any one or more of the methodologies or functions described herein. A non-transitory storage medium refers to a storage medium other than a carrier wave. The instructions 1226 may also reside, completely or at least partially, within the main memory 1204 and/or within the processing device 1202 during execution thereof by the computer device 1200, the main memory 1204 and the processing device 1202 also constituting computer-readable storage media.

The computer-readable storage medium 1224 may also be used to store a dental condition determiner 1250, which may correspond to the similarly named component of FIG. 1. The computer readable storage medium 1224 may also store a software library containing methods for a dental condition determiner 1250. While the computer-readable storage medium 1224 is shown in an example embodiment to be a single medium, the term “computer-readable storage medium” should be taken to include a single medium or multiple media (e.g., a centralized or distributed database, and/or associated caches and servers) that store the one or more sets of instructions. The term “computer-readable storage medium” shall also be taken to include any non-transitory medium (e.g., a medium other than a carrier wave) that is capable of storing or encoding a set of instructions for execution by the machine and that cause the machine to perform any one or more of the methodologies of the present disclosure. The term “computer-readable storage medium” shall accordingly be taken to include, but not be limited to, solid-state memories, and optical and magnetic media.

It is to be understood that the above description is intended to be illustrative, and not restrictive. Many other embodiments will be apparent upon reading and understanding the above description. Although embodiments of the present disclosure have been described with reference to specific example embodiments, it will be recognized that the disclosure is not limited to the embodiments described, but can be practiced with modification and alteration within the spirit and scope of the appended claims. Accordingly, the specification and drawings are to be regarded in an illustrative sense rather than a restrictive sense. The scope of the disclosure should, therefore, be determined with reference to the appended claims, along with the full scope of equivalents to which such claims are entitled.

What is claimed is:

1. A computer readable medium comprising instructions that, when executed by a processing device, cause the processing device to perform operations comprising:
receiving an image of dental site of a patient;
estimating a presence of one or more dental conditions in the image;
estimating a severity level of each of the one or more dental conditions at one or more locations in the image;
generating a severity map of the one or more dental conditions for the image based on the estimated presence of the one or more dental conditions and the estimated severity level of each of the one or more dental conditions at the one or more locations in the image; and
projecting the severity map of the one or more dental conditions for the image onto a model of the dental site to generate a projected severity map of the one or more dental conditions.

2. The computer readable medium of claim 1, wherein the model is a 3D model and the projected severity map is a 3D severity map, the operations further comprising:
presenting the 3D model of the dental site together with the 3D severity map of the one or more dental conditions in a graphical user interface (GUI).

3. The computer readable medium of claim 1, wherein estimating the presence of the one or more dental conditions in the image comprises processing the image using one or more trained machined learning models, wherein each of the one or more trained machine learning models outputs a probability of the dental site containing the one or more dental conditions.

4. The computer readable medium of claim 1, wherein the model is a 3D model of the dental site, the operations further comprising:
receiving a plurality of intraoral scans of the dental site; and
generating the 3D model of the dental site using the plurality of intraoral scans.

5. The computer readable medium of claim 1, the operations further comprising:
receiving an intraoral scan of the dental site that is associated with the image;
wherein the presence of the one or more dental conditions is estimated by inputting the image and the intraoral scan into a trained machine learning model that outputs a probability of the dental site containing the one or more dental conditions.

6. The computer readable medium of claim 1, wherein the one or more dental conditions are selected from a group consisting of caries, gum recession, gum inflammation, tooth wear, malocclusion, tooth crowding, tooth spacing, plaque, tooth stains, and tooth cracks.

7. The computer readable medium of claim 1, wherein the image comprises one of a two-dimensional (2D) color image generated by an intraoral scanner, a 2D near infrared image generated by the intraoral scanner, or a 2D color image generated by an image sensor of a device other than an intraoral scanner.

8. The computer readable medium of claim 1, wherein estimating the severity level of each of the one or more dental conditions at one or more locations in the image comprises:
taking a derivative of the estimation with respect to input pixel intensities of the image, wherein severity is a function of the derivative.

9. The computer readable medium of claim 1, wherein estimating the severity level of each of the one or more dental conditions at one or more locations in the image comprises:
erasing a region of the image;
generating a modified image by processing the image with the erased region by a machine learning model trained to generate images of healthy dental sites, wherein the machine learning model fills in data for the erased region of the image;
estimating the presence of the one or more dental conditions in the modified image; and
determining a change in the estimation of the presence of the one or more dental conditions between the modified image and the image.

10. The computer readable medium of claim 9, wherein the machine learning model is a generative adversarial network (GAN).

11. The computer readable medium of claim 1, wherein estimating the severity level of each of the one or more dental conditions at one or more locations in the image comprises:
inputting the image into a trained machine learning model that generates a feature vector representing the image and reconstructs the image from the feature vector, the trained machine learning model having been trained on images lacking the one or more dental conditions;
determining differences between the image and the reconstructed image; and
generating the severity map based on the differences between the image and the reconstructed image.

12. The computer readable medium of claim 11, wherein for each location of the image a degree of difference between the image and the reconstructed image at the location provides the severity level of one or more dental conditions at the location.

13. The computer readable medium of claim 1, wherein the image is a near infrared image and the one or more dental conditions comprises caries.

14. The computer readable medium of claim 1, wherein the image is a color image and the one or more dental conditions comprises gum inflammation.

15. The computer readable medium of claim 1, further comprising:
separately estimating, for each dental condition of a plurality of dental conditions, the presence of the dental condition in the image and the severity level of the dental condition at the one or more locations in the image;
generating a plurality of severity maps for the image, wherein each of the plurality of severity maps is associated with a different one of the plurality of dental conditions; and
projecting each of the plurality of severity maps onto the model of the dental site to generate a plurality of projected severity maps, each associated with the different one of the plurality of dental conditions.

16. The computer readable medium of claim 15, further comprising:
generating a combined projected severity map based on the plurality of projected severity maps, wherein for each location of the one or more locations a combined severity level is determined based on severity levels of each of the plurality of projected severity maps at the location.

17. The computer readable medium of claim 16, further comprising:
presenting the combined projected severity map overlaid on the model in a graphical user interface GUI);
receiving a selection of a location of the one or more locations; and
presenting separate data for one or more of the plurality of projected severity maps at the selected location.

18. The computer readable medium of claim 15, further comprising:
presenting the projected model of the dental site;
receiving a selection of a dental condition of interest; and
presenting a projected severity map of the plurality of projected severity maps that is associated with the selected dental condition of interest.

19. The computer readable medium of claim 1, wherein estimating the severity level of each of the one or more dental conditions at one or more locations in the image is performed using a gradient-weighted class activation mapping (Grad-CAM) algorithm.

20. The computer readable medium of claim 1, wherein the severity map comprises a heat map.

21. The computer readable medium of claim 1, wherein estimating the severity level of each of the one or more dental conditions at one or more locations in the image comprises:
dividing the image into a plurality of overlapping patches, wherein each pixel of the image contributes to more than one of the plurality of overlapping patches;
for each patch of the plurality of overlapping patches, processing the patch using a model that outputs a probability of that patch containing the one or more dental conditions; and
for each pixel of the image, determining a severity level of the one or more dental conditions at the pixel based on a combination of probabilities of patches that include the pixel containing the one or more dental conditions.

22. The computer readable medium of claim 1, wherein the image comprises a first time stamp, the operations further comprising:
receiving a second image that comprises a second time stamp that predates the first time stamp;
comparing the projected severity map to a second projected severity map generated for the second image to determine differences therebetween; and
determining rates of change in severity levels for the one or more dental conditions based on a result of the comparing.

23. The computer readable medium of claim 22, the operations further comprising:
identifying one or more locations at which a rate of change of the severity level exceeds a rate of change threshold; and
flagging the one or more locations.

24. The computer readable medium of claim 22, the operations further comprising:
determining a recommended frequency for the patient to visit a dentist based on at least one of the severity level or the rate of change of the severity level for the one or more dental conditions.

25. The computer readable medium of claim 1, the operations further comprising:
identifying one or more locations at which the severity level exceeds a severity threshold; and
flagging the one or more locations.

26. The computer readable medium of claim 1, the operations further comprising:
generating, based on the estimated severity level of the one or more dental conditions at the one or more locations in the image, a recommendation for a dentist to assess the dental site.

27. The computer readable medium of claim 1, the operations further comprising:
generating a plurality of additional projected severity maps of the one or more dental conditions from a plurality of additional images; and
resolving differences between the projected severity map and the plurality of additional projected severity maps using a voting algorithm.

28. The computer readable medium of claim 1, wherein the model comprises a panoramic image of the dental site.

29. A system comprising:
a memory; and
a processing device operatively connected to the memory, the processing device to:
receive an image of dental site of a patient;
estimate a presence of one or more dental conditions in the image;
estimate a severity level of each of the one or more dental conditions at one or more locations in the image;
generate a severity map of the one or more dental conditions for the image based on the estimated presence of the one or more dental conditions and the estimated severity level of each of the one or more dental conditions at the one or more locations in the image; and
project the severity map of the one or more dental conditions for the image onto a model of the dental site to generate a projected severity map of the one or more dental conditions.

30. A method comprising:
receiving an image of dental site of a patient;
estimating a presence of one or more dental conditions in the image;
estimating a severity level of each of the one or more dental conditions at one or more locations in the image;
generating a severity map of the one or more dental conditions for the image based on the estimated presence of the one or more dental conditions and the estimated severity level of each of the one or more dental conditions at the one or more locations in the image; and
projecting the severity map of the one or more dental conditions for the image onto a model of the dental site to generate a projected severity map of the one or more dental conditions.
